(19) United States
(12) Patent Application Publication
(10) Pub. No.: US 2025/0034117 A1
(43) Pub. Date: Jan. 30, 2025

(54) Deuterated 1,3 Dihydro -2H-indole-2-one derivatives

(71) Applicant: HMNC Holding GmbH, München (DE)

(72) Inventors: Hans ERIKSSON, München (DE); Werner STÜBER, München (DE)

(21) Appl. No.: 18/771,709

(22) Filed: Jul. 12, 2024

Related U.S. Application Data
(60) Provisional application No. 63/513,158, filed on Jul. 12, 2023.

(30) Foreign Application Priority Data
Jul. 12, 2023 (EP) 23185135.3

(51) Int. Cl. C07D 403/04; A61K 31/404

(52) U.S. Cl. CPC C07D 403/04; A61K 31/404

(57) ABSTRACT

The present invention relates to deuterated 1,3 Dihydro-2H-indole-2-one derivatives and a pharmaceutical composition comprising said deuterated compounds. It further relates to a method of manufacturing said deuterated compounds and the use of said compounds as a medicament. The present invention also relates to intermediates suitable for the preparation of the compounds of the invention.

### CROSS REFERENCE TO RELATED APPLICATIONS

This application claims the benefit priority to U.S. Provisional Patent Application Ser. No. 63/513,158, filed Jul. 12, 2023, and European Patent Application Serial No. EP 23185135.3, filed Jul. 12, 2023, which are both incorporated by reference herein in their entirety.

### FIELD

The present invention relates to deuterated 1,3 Dihydro-2H-indole-2-one derivatives and a pharmaceutical composition comprising said deuterated compounds. It further relates to a method of manufacturing said deuterated compounds and the use of said compounds as a medicament, in particular for use in the treatment or prophylaxis of anxiety or depression. The present invention also relates to intermediates suitable for the preparation of the compounds of the invention.

### BACKGROUND OF THE INVENTION

1,3-dihydro-2H indol-2-one derivatives having affinity for and selectivity towards the V_{1b} receptor or towards both the V₁ₐ and V_{1b} receptors of arginine vasopressin (AVP) are known in the art, e.g. from U.S. Pat. No. 6,730,695 B2. AVP is a hormone, which is known for its antidiuretic effect and its effect in regulating arterial pressure. It stimulates several types of receptors: V₁ (V₁ₐ, V_{1b}), and V₂. These receptors are located in particular in the liver, the vessels (coronary, renal and cerebral), the platelets, the kidneys, the uterus, the adrenal glands, the pancreas, the central nervous system and the pituitary gland. AVP thus exerts cardiovascular, hepatic, pancreatic, antidiuretic and platelet aggregating effects and effects on the central and peripheral nervous system, and on the uterine sphere. In U.S. Pat. No. 6,730,695 B2, 1,3-dihydro-2H indol-2-one derivatives were reported to be used for the preparation of medicinal products that are useful in the treatment or prevention of any pathology in which arginine-vasopressin and/or the V_{1b} receptors or both the V₁ₐ receptors and the V_{1b} receptors are involved. In particular, the compounds are suggested for the treatment or prevention of disorders of the cardiovascular system, for example hypertension; of the central nervous system, for example stress, anxiety, depression, compulsive obsessive disorder and panic attacks, of the renal system; of the gastric system as well as in the treatment of small cell lung cancers, of obesity; of type II diabetes, of insulin resistance, of hypertriglyceridemia; of atherosclerosis, of Cushing's Syndrome; of any pathology following stress and chronic stress states.

Nelivaptan (SSR-149,415; (2S,4R)-1-[(3R)-5-chloro-1-(2,4-dimethoxyphenyl) sulfonyl-3-(2-methoxyphenyl)-2-oxoindol-3-yl]-4-hydroxy-N,N-dimethylpyrrolidine-2-carboxamide) is a selective, orally active, non-peptide vasopressin receptor antagonist selective for the V_{1b} subtype. The drug had entered clinical trials for treatment of anxiety and depression. Nelivaptan and a process for the preparation thereof is disclosed in U.S. Pat. No. 6,730,695 B2. Amorphous nelivaptan is disclosed in WO2009140484 A1.

Nelivaptan has a relatively short degradation half-life; at 10 mg/kg p.o. its duration of action is reported as being “longer than 4 h” (C. Serradeil-Le Gal et al, CNS Drug Reviews 2005, Vol. 11, No. 1, pp. 53-68).

It would be desirable to provide vasopressin receptor antagonists selective for the V_{1b} subtype having a longer degradation half-life, e.g. in order to allow less frequent dosing.

While generally methods to increase the degradation half-life of pharmaceutical agents, like adding halogen atoms (e.g. Gunaydin et al, ACS Med. Chem. Lett. 2018, 9, 528-533), are known in the art, none of the methods is reliable and rather all methods are based on trial and error.

Applicants have discovered that deuterated 1,3 dihydro-2H-indole-2-one derivatives have surprisingly a significantly increased degradation half-life, which thus allows less frequent dosing.

### SUMMARY OF THE INVENTION

In a first aspect, the present invention relates to a compound of formula (I):

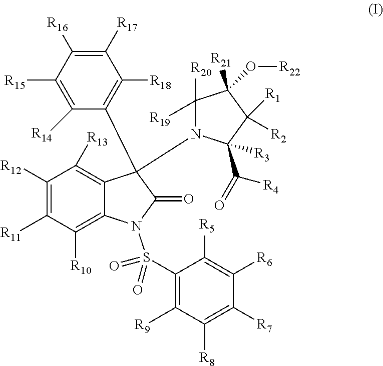

or a pharmaceutically acceptable salt or prodrug thereof, wherein:
R₁, R₂, R₃, R₁₉, R₂₀, and R₂₁ are individually selected from the group consisting of hydrogen, C₁₋₄ alkyl, and C₁₋₄ alkoxy,
R₅, R₆, R₇, R₈, R₉, R₁₀, R₁₁, R₁₂, R₁₃, R₁₄, R₁₅, R₁₆, R₁₇, and R₁₈ are individually selected from the group consisting of hydrogen, halogen, C₁₋₄ alkyl, C₁₋₄ haloalkyl, C₁₋₄ alkoxy, and C₁₋₄ haloalkoxy,
R₄ is selected from the group consisting of C₁₋₄ alkylamino, C₁₋₄ haloalkylamino, di-C₁₋₄ alkylamino, di-C₁₋₄ haloalkylamino, and azetidin-1-yl,
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀,
R₃₈ is selected from the group consisting of hydrogen, hydroxy and C₁₋₄ alkyl,
R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and
integer n is 1 or 2, provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ is enriched in deuterium.

In an embodiment of the first aspect, the compound comprises at least an amount of deuterium as follows:
a) at least one hydrogen of R₄, which is selected from C₁₋₄ alkylamino or di-C₁₋₄ alkylamino, is enriched in deuterium, and/or
b) at least one hydrogen of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium.
   Preferably, both a) and b) are fulfilled.

In an embodiment of the first aspect,
a) at least one hydrogen, preferably all hydrogens, of R₄, which is selected from C₁₋₄ alkylamino or di-C₁₋₄ alkylamino, is enriched in deuterium, and/or
b) at least one hydrogen, preferably all hydrogens, of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium. Preferably, both a) and b) are fulfilled.

In a second aspect, the present invention provides a process for the preparation of a compound of formula (I):

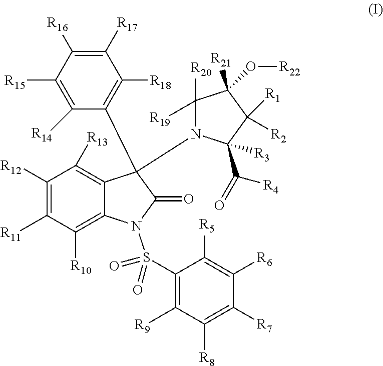

or a pharmaceutically acceptable salt or prodrug thereof, wherein:
R₁, R₂, R₃, R₁₉, R₂₀, and R₂₁ are individually selected from the group consisting of hydrogen, C₁₋₄ alkyl, and C₁₋₄ alkoxy,
R₅, R₆, R₇, R₈, R₉, R₁₀, R₁₁, R₁₂, R₁₃, R₁₄, R₁₅, R₁₆, R₁₇, and R₁₈ are individually selected from the group consisting of hydrogen, halogen, C₁₋₄ alkyl, C₁₋₄ haloalkyl, C₁₋₄ alkoxy, and C₁₋₄ haloalkoxy,
R₄ is selected from the group consisting of C₁₋₄ alkylamino, C₁₋₄ haloalkylamino, di-C₁₋₄ alkylamino, di-C₁₋₄ haloalkylamino, and azetidin-1-yl,
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀,
R₃₈ is selected from the group consisting of hydrogen, hydroxy and C₁₋₄ alkyl,
R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and
integer n is 1 or 2, provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ is enriched in deuterium,
preferably wherein
a) at least one hydrogen of R₄, which is selected from C₁₋₄ alkylamino or di-C₁₋₄ alkylamino, is enriched in deuterium, and/or
b) at least one hydrogen of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium,
the process comprising a step of reacting at least one of the following compounds of formulae (VI), (VII), (VIII), or (IX)

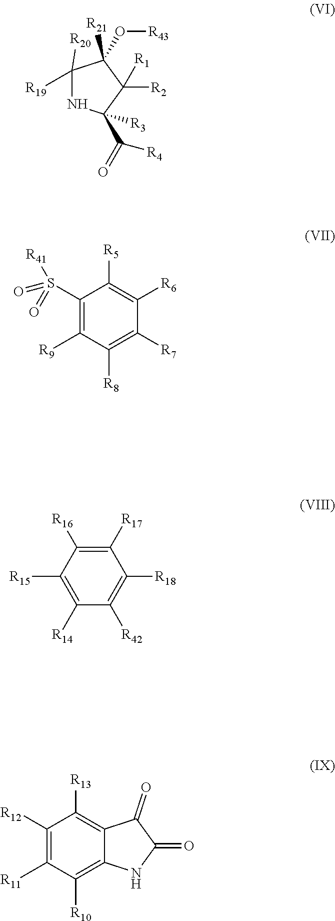

wherein R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁ are as defined for formula (I), R₄₁ is a halogen or an activating ester residue, R₄₂ is a halogen, and R₄₃ is selected from the group consisting of hydrogen, C₁₋₄ alkyl, C₁₋₄ haloalkyl, —(CH₂)ₙ—CO—R₃₈, —CO—(CH₂)ₙ—NR₃₉R₄₀, and a hydroxyl protecting group, provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ of the compound of formulae (VI), (VII), (VIII), or (IX) used is enriched in deuterium, wherein preferably
a) at least one hydrogen of R₄, which is selected from C₁₋₄ alkylamino or di-C₁₋₄ alkylamino, is enriched in deuterium, and/or
b) at least one hydrogen of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium.

In a third aspect of the present invention relates to a pharmaceutical composition comprising a compound according to formula (I) according to the first aspect of the present invention and a pharmaceutical acceptable carrier.

In a fourth aspect of the present invention relates to a compound according to formula (I) according to the first aspect or a pharmaceutical composition according to the third aspect of the present invention for use as medicament.

In a fifth aspect, the present invention relates to a compound according to formula (I) according to the first aspect or a pharmaceutical composition according to the third aspect of the present invention for the treatment or prophylaxis of disorders of the central nervous system, in particular anxiety or depression.

In a sixth aspect, the present invention relates to a compound according to formula (X):

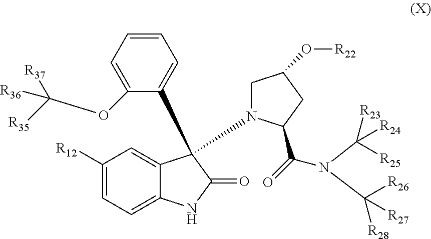

wherein R₁₂ is selected from the group consisting of hydrogen, halogen, C₁₋₄ alkyl, C₁₋₄ haloalkyl, C₁₋₄ alkoxy, and C₁₋₄ haloalkoxy, preferably a methyl, trifluoromethyl or halogen, more preferably chlorine, R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀, R₃₈ is selected from the group consisting of hydrogen, hydroxy and C₁₋₄ alkyl, R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and integer n is 1 or 2, or R₂₂ is a hydroxyl protecting group, and R₂₃, R₂₄, R₂₅, R₂₆, R₂₇, R₂₈, R₃₅, R₃₆ and R₃₇ are hydrogen, provided that at least one, preferably at least two or at least three, of R₂₃, R₂₄, R₂₅, R₂₆, R₂₇, R₂₈, R₃₅, R₃₆ and R₃₇ is enriched in deuterium.

### DESCRIPTION OF THE FIGURES

In the following, the content of the Figures comprised in this specification is described. In this context, it is also referred to the detailed description of the invention above and/or below.

FIG. 1: Example for the general synthesis scheme of 1,3 dihydro-2H-indole-2-one derivatives applicable to the compounds of the invention. In the exemplified synthesis of the compounds of the invention shown in FIG. 1, Compound 1 is converted into Compound 2, i.e. a corresponding amine derivative. The latter is reacted with Compound 6, which is available by reacting Compounds 3 and 4 to obtain Compound 5, which can be transferred to obtain Compound 6. The reaction of Compounds 2 and 6 results in Compound 7, which can be further reacted with Compound 8 to obtain the final product of Compound 9. In order to obtain deuterated compounds according to the invention, deuterated starting materials, e.g. deuterated Compounds 1, 2, 3, 4, or 8 may be used.

FIG. 2: Comparison of degradation half-life of the compounds of the invention relative to the degradation half-life of undeuterated nelivaptan. The compounds of the invention show an increased degradation half-life in the degradation test as described in Example 5 compared to the degradation half-life of undeuterated nelivaptan.

FIG. 3: 1H-NMR in CDCl₃ of (2S,4R)1-[(3R)-5-chloro-1-[(2,4-dimethoxyphenyl) sulfonyl]-2,3-dihydro-3-(2-methoxyphenyl)-2-oxo-1H-indol-3-yl]-4-hydroxy-N,N-dimethyl-d6-2-pyrrolidinecarboxamide (Compound of Example 1).

FIG. 4: 1H-NMR in CDCl₃ of (2S,4R)1-[(3R)-5-chloro-1-[(2,4-dimethoxyphenyl) sulfonyl]-2,3-dihydro-3-(2-methoxy-d3-phenyl)-2-oxo-1H-indol-3-yl]-4-hydroxy-N,N-dimethyl-2-pyrrolidinecarboxamide (Compound of Example 2).

FIG. 5: 1H-NMR in CDCl₃ of (2S,4R)1-[(3R)-5-chloro-1-[(2,4-dimeth-d6-oxyphenyl) sulfonyl]-2,3-dihydro-3-(2-methoxy-d3-phenyl)-2-oxo-1H-indol-3-yl]-4-hydroxy-N,N-dimethyl-d6-2-pyrrolidinecarboxamide (Compound of Example 3).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions, etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

As used in this specification and the appended claims, the singular forms “a”, “an”, and “the” include plural referents, unless the content clearly dictates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step.

The term “alkyl”, by itself or as part of another substituent, means, unless otherwise stated, a straight or branched chain hydrocarbon radical, having the number of carbon atoms designated (i.e. C₁₋₄ means one to four carbons). Preferred examples of C₁₋₄ alkyl mean methyl, ethyl, n-propyl, isopropyl, n-butyl and tert-butyl. More preferred, C₁₋₄ alkyl is a methyl group or an ethyl group, in particular a methyl group.

The terms “alkoxy”, “alkylamino”, or “dialkylamino” are used in their conventional sense, and refer to those alkyl groups attached to the remainder of the molecule via an oxygen atom, or an amino group respectively. Additionally, for dialkylamino groups, the alkyl portions can be the same or different and can also be combined to form a 3-7 membered ring with the nitrogen atom to which each is attached. Preferred examples of C₁₋₄ alkoxy mean methoxy, ethoxy, n-propoxy, isopropoxy, n-butyloxy and tert-butyloxy. More preferred, C₁₋₄ alkoxy is a methoxy group or an ethoxy group, in particular a methoxy group. Preferred examples of C₁₋₄ alkylamino mean methylamino, ethylamino, n-propylamino, isopropylamino, n-butylamino and tert-butyl amino. More preferred, C₁₋₄ alkylamino is a methylamino or an ethylamino group, in particular a methylamino group. Preferred examples of di-C₁₋₄ alkylamino mean dimethylamino, diethylamino, methylethylamino, di-n-propylamino, di-isopropylamino, di-n-butylamino and di-tert-butylamino. More preferred, di-C₁₋₄ alkylamino is a dimethylamino or a diethylamino group, in particular a dimethylamino group.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, pentafluoroethyl, or trichloromethyl, trifluoromethyl being preferred.

The term “hydroxyl protecting group” means a group introduced into a molecule by chemical modification of a functional group, namely hydroxy group, to obtain chemoselectivity in a subsequent chemical reaction. Suitable hydroxyl protecting groups as well as the conditions for adding and removing them are known in the art. Examples of hydroxyl protecting groups are acetyl (Ac), benzoyl (Bz), benzyl (Bn), methoxyethoxymethyl ether (MEM), dimethoxytrityl, [bis-(4-methoxyphenyl)phenylmethyl] (DMT), methoxymethyl ether (MOM), methoxytrityl [(4-methoxyphenyl)diphenylmethyl] (MMT), p-Methoxybenzyl ether (PMB), p-methoxyphenyl ether (PMP), methylthiomethyl ether, pivaloyl (Piv), tert-butyl ethers (tBu), tetrahydropyranyl (THP), tetrahydrofuran (THF), trityl (triphenylmethyl, Tr), silyl ether (most preferred ones include trimethylsilyl (TMS), tert-butyldimethylsilyl (TBDMS or TBS), tri-iso-propylsilyloxymethyl (TOM), and triisopropylsilyl (TIPS) ethers), methyl ethers, and ethoxyethyl ethers.

The term “activating ester residue” means as known in the art, an active ester, i.e. an ester functional group that is highly susceptible toward nucleophilic attack. Herein, activation can be imparted by modifications of the substituent at the sulfonate compound of formula (VII) in order to obtain a good leaving group such that the ester can be reacted with an amine compound or NH-containing cyclic compound to obtain the corresponding N-sulfonyl derivative, e.g. a compound of formula (I). Examples of “activating ester residue” are known reaction products with N-hydroxysuccinimide, hydroxybenzotriazole, or ethyl 2-cyano-2-(hydroxyimino)acetate (Oxyma).

“Pharmaceutically acceptable” means approved by a regulatory agency of the Federal or a state government or listed in the U.S. Pharmacopeia (United States Pharmacopeia-33/National Formulary-28 Reissue, published by the United States Pharmacopeia Convention, Inc., Rockville Md., publication date: April 2010) or other generally recognized pharmacopeia for use in animals, and more particularly in humans.

The term “pharmaceutically acceptable salt” refers to a salt of the compounds according to formula (I) as described herein. Suitable pharmaceutically acceptable salts include acid addition salts which may, for example, be formed by mixing a solution of a of the compounds according to formula (I) or a positively or negatively charged derivative thereof with a solution of a pharmaceutically acceptable acid. The pharmaceutically acceptable salts of the compounds of formula (I) are, for example, the hydrochloride, hydrobromide, sulphate, hydrogen sulphate, dihydrogen phosphate, methanesulphonate, benzenesulphonate, naphthalenesulphonate, paratoluenesulphonate, maleate, fumarate, succinate, citrate, acetate, gluconate or oxalate.

The neutral forms of the compounds according to formula (I) as described herein may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the compounds according to formula (I) as described herein can be used in a prodrug form. Prodrugs of the compounds according to formula (I) are those compounds that readily undergo chemical changes under physiological conditions to provide the compounds according to formula (I), respectively. A prodrug is an active or inactive compound that is modified chemically through in vivo physiological action, such as hydrolysis, metabolism and the like, into a compound of this invention following administration of the prodrug to a patient. Additionally, prodrugs can be converted to the compounds used in the present invention by chemical or biochemical methods in an ex vivo environment. The suitability and techniques involved in making and using prodrugs are well known by those skilled in the art. For a general discussion of prodrugs involving esters, see Svensson L. A. and Tunek A. (1988) Drug Metabolism Reviews 19 (2): 165-194 and Bundgaard H. “Design of Prodrugs”, Elsevier Science Ltd. (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives, which are cleaved by esterases in vivo releasing the free drug and formaldehyde (Bundgaard H. et al. (1989) J. Med. Chem. 32 (12): 2503-2507). Also, drugs containing an acidic NH group, such as imidazole, imide, indole and the like, have been masked with N-acyloxymethyl groups (Bundgaard H. “Design of Prodrugs”, Elsevier Science Ltd. (1985)). Hydroxy groups have been masked as esters and ethers. EP 0 039 051 A2 discloses Mannich-base hydroxamic acid prodrugs, their preparation and use.

The compounds according to formula (I) as described herein may also contain unnatural proportions of atomic isotopes, other than deuterium, at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds according to formula (I) as described herein, whether radioactive or not, are intended to be encompassed within the scope of the present invention.

As used herein, a “patient” means any mammal or bird that may benefit from a treatment with the compounds described herein. Preferably, a “patient” is selected from the group consisting of laboratory animals, domestic animals, or primates including chimpanzees and human beings. It is particularly preferred that the “patient” is a human being.

As used herein, “treat”, “treating” or “treatment”, or ameliorating, respectively, of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in a subject for a certain amount of time. For example, if a compound described herein is administered to a subject with the aim of preventing a disease or disorder, said disease or disorder is prevented from occurring at least on the day of administration and preferably also on one or more days (e.g. on 1 to 30 days; or on 2 to 28 days; or on 3 to 21 days; or on 4 to 14 days; or on 5 to 10 days) following the day of administration.

A “pharmaceutical composition” according to the invention may be present in the form of a composition, wherein one or more active ingredient and one or more diluents and/or carriers are admixed with each other, or may take the form of a combined preparation, where active ingredients are present in partially or totally distinct form. An example for such a combination or combined preparation is a kit-of-parts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, and the like. The composition can be formulated as a suppository, with traditional binders and carriers such as triglycerides. The compounds of the invention can be formulated as neutral or salt forms. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

The term “enriched in deuterium” as used within this specification means for the referenced substituent group or groups that comprise hydrogen in the compounds, instead of comprising naturally-occurring hydrogen, the referenced groups(s) comprise deuterium in more than its natural abundance. Deuterium has a natural abundance of about 0.0156%. Accordingly, for approximately every 6,500 hydrogen atoms occurring in nature, there is one deuterium atom. Disclosed herein are compounds enriched in deuterium at one or more positions. Thus, deuterium containing compounds of the disclosure have deuterium at one or more positions (as the case may be) in an abundance of greater than 0.0156%. So, as used herein, when a moiety or compound is described as being enriched in deuterium or as having a deuterium present, it is meant that there is an abundance in deuterium at the position at which the deuterium is substituted or present to of greater than 0.0156%. Preferably, the enrichment is at least 10%, at least 30%, at least 50%, at least 70%, at least 97%, at least 98%, or at least 99%. Most preferably, the respective hydrogen is substantially replaced by deuterium, in particular is replaced/substituted by deuterium. When, as used herein, at least one hydrogen of a residue is enriched in deuterium, this covers both the case where the residue represents a hydrogen (e.g., “H” which is enriched in deuterium) and the case where the residue carries one or more hydrogen atoms (e.g., “—N(CH₃) 2”) in which at least one or up to all hydrogen is/are enriched in deuterium).

The term “depression” is a clinical symptom including persistent feelings of sadness, hopelessness and/or the lose interest in activities. To be diagnosed with depression symptoms persist for at least two weeks without treatment. Symptoms of major depression are typically measured using a rating scale such as the Hamilton depression rating scale for depression (HAM-D) or the Montgomery Asberg depression rating scale (MADRS). Major depression may also be associated with anxiety symptoms, which may be measured using a rating scale the Hamilton depression rating Scale (HAM-A). Depression may be classified as major depressive disorder (MDD) or bipolar depression (BPD) and can be diagnosed using the criteria described in the Diagnostic and Statistical Manual of Mental Disorders, 5th ed. (DSM-5-TR) published by the American Psychiatric Association, which also provides additional description of psychiatric disorders or may be diagnosed according to standards described in the International classification of diseases 11 (ICD-11).

The term “anxiety” or “anxiety disorder” refers to a state of anxiety, uncertainty, and/or fear that may arise from the expectations of an event and/or situation. Anxiety can be diagnosed using the criteria described in the Diagnostic and Statistical Manual of Mental Disorders, 5th ed. (DSM-5-TR) published by the American Psychiatric Association, which also provides additional description of psychiatric disorders or may be diagnosed according to standards described in the international classification of diseases 11 (ICD-11).

### Embodiments of the Invention

In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. In the work leading to the present invention, it was shown that deuterated 1,3 dihydro-2H-indole-2-one derivatives according to formula (I) surprisingly have significantly increased degradation half-lifes, which thus allows less frequent dosing.

Based on these results, the present invention provides in a first aspect a compound of formula (I):

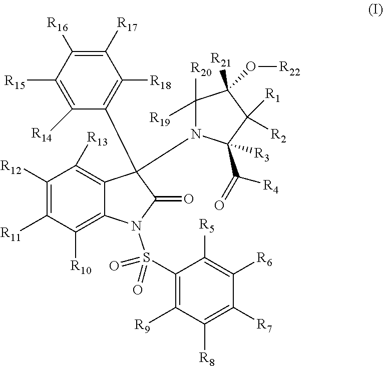

or a pharmaceutically acceptable salt or prodrug thereof, wherein:
R₁, R₂, R₃, R₁₉, R₂₀, and R₂₁ are individually selected from the group consisting of hydrogen, C₁₋₄ alkyl, and C₁₋₄ alkoxy, preferably hydrogen;
R₅, R₆, R₇, R₈, R₉, R₁₀, R₁₁, R₁₂, R₁₃, R₁₄, R₁₅, R₁₆, R₁₇, and R₁₈ are individually selected from the group consisting of hydrogen, halogen, C₁₋₄ alkyl, C₁₋₄ haloalkyl, C₁₋₄ alkoxy, and C₁₋₄ haloalkoxy, preferably R₆, R₈, R₉, R₁₀, R₁₁, R₁₃, R₁₅, R₁₆, R₁₇, and R₁₈ are hydrogen, R₁₂ is preferably halogen, C₁₋₄ alkyl, or C₁₋₄ haloalkyl, most preferably halogen, and R₅, R₇, and R₁₄, are preferably C₁₋₄ alkoxy, more preferably methoxy;
R₄ is selected from the group consisting of C₁₋₄ alkylamino, C₁₋₄ haloalkylamino, di-C₁₋₄ alkylamino, di-C₁₋₄ haloalkylamino, and azetidin-1-yl, preferably R₄ is C₁₋₄ alkylamino or di-C₁₋₄ alkylamino, in particular dimethylamino;
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀, preferably hydrogen; R₃₈ is selected from the group consisting of hydrogen, hydroxy and C₁₋₄ alkyl, R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and integer n is 1 or 2, provided that at least one hydrogen of residues R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ is enriched in deuterium, wherein preferably
a) at least one hydrogen of R₄, which is selected from C₁₋₄ alkylamino or di-C₁₋₄ alkylamino, is enriched in deuterium, and/or
b) at least one hydrogen of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium.

Preferably, the compound comprises at least an amount of deuterium as follows:
a) at least one hydrogen, preferably all hydrogens, of R₄, which is selected from C₁₋₄ alkylamino or di-C₁₋₄ alkylamino, is enriched in deuterium, and/or
b) at least one hydrogen, preferably all hydrogens, of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium. Preferably, both a) and b) are fulfilled.

That is, in the compounds of the present invention, in particular in the compounds of formula (I), at least one hydrogen of any one of the residues R₁ to R₂₁ (preferably at least one hydrogen of R₄, R₅, R₇, or R₁₄ and optionally additionally any one of the other residues R₁, R₂, R₃, R₆, R₈, R₉, R₁₀, R₁₁, R₁₃, R₁₅, R₁₆, R₁₇, R₁₈, R₁₉, R₂₀, or R₂₁), i.e. a hydrogen as part of the residue, or hydrogen constituting said residue, is enriched in deuterium, meaning that the hydrogen atoms (H₁) at the respective position is replaced by deuterium (H₂) at an abundance more than naturally occurring, i.e. of greater than about 0.0156%. Preferably, the enrichment is at least 10%, at least 30%, at least 50%, at least 70%, at least 97%, at least 98%, or at least 99%. That is, preferably, at a respective position of the residue, or as the residue as such, at least 10%, at least 30%, at least 50%, at least 70%, at least 97%, at least 98%, or at least 99% of the hydrogen is replaced by deuterium.

In preferred embodiments, in the compounds according to formula (I) of the present invention,
a) at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium,
b) at least one hydrogen and preferably all hydrogens of R₄, which is selected from C₁₋₄ alkylamino or di-C₁₋₄ alkylamino, preferably dimethylamino, is enriched in deuterium, and/or
c) at least one hydrogen and preferably all hydrogens of R₅, R₇, or R₁₄, which are individually selected from C₁₋₄ alkyl or C₁₋₄ alkoxy, preferably methoxy, is enriched in deuterium.

Preferably, in the compounds of formula (I), a), b) and c) are fulfilled. That is, in the compounds according to formula (I), preferably at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium, at least one hydrogen and preferably all hydrogens of R₄, which is selected from C₁₋₄ alkylamino or di-C₁₋₄ alkylamino, preferably dimethylamino, is enriched in deuterium, and at least one hydrogen and preferably all hydrogens of R₅, R₇, and/or R₁₄, which are individually selected from C₁₋₄ alkyl or C₁₋₄ alkoxy, preferably methoxy, is enriched in deuterium.

In a particularly preferred embodiment, in the compounds according to formula (I) of the present invention,
a) at least one hydrogen and preferably all hydrogens of R₄, which is selected from C₁₋₄ alkylamino or di-C₁₋₄ alkylamino, preferably dimethylamino, is enriched in deuterium, and/or
b) at least one hydrogen and preferably all hydrogens of R₅, R₇, or R₁₄, which are individually selected from C₁₋₄ alkyl or C₁₋₄ alkoxy, preferably methoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium.

In further preferred embodiments, in the compounds according to formula (I) of the present invention,
d) R₅, R₇, and R₁₄ are a methoxy group,
e) R₁₂ is methyl, trifluoromethyl or halogen, preferably halogen, more preferably a chlorine atom,
f) R₂₂ is hydrogen, and/or
g) R₁, R₂, R₃, R₆, R₈, R₉, R₁₀, R₁₁, R₁₃, R₁₅, R₁₆, R₁₇, R₁₈, R₁₉, R₂₀, and R₂₁ are hydrogen.

Preferably, in the compounds of formula (I), d), e), f) and g) are fulfilled. That is, in the compounds according to formula (I), preferably R₅, R₇, and R₁₄ are a methoxy group, R₁₂ is halogen, preferably a chlorine atom, R₂₂ is hydrogen, and R₁, R₂, R₃, R₆, R₈, R₉, R₁₀, R₁₁, R₁₃, R₁₅, R₁₆, R₁₇, R₁₈, R₁₉, R₂₀, and R₂₁ are hydrogen. In this embodiment, preferably at least one hydrogen and preferably all hydrogens of R₅, R₇, and/or R₁₄, is enriched in deuterium.

In a preferred embodiment, in the compounds of formula (I), at least one hydrogen, preferably all six hydrogens, of R₄, which is dimethylamino, and/or at least one hydrogen, preferably all three hydrogens, of R₅, R₇, or R₁₄, which are a methoxy group, is enriched in deuterium. In one embodiment, in the compounds of formula (I), all six hydrogens, of R₄, which is dimethylamino, and all three hydrogens of each of R₅ and R₇, or of R₅ and R₁₄, or of R₇ and R₁₄, which are all a methoxy group, are enriched in deuterium.

In one embodiment, in the compounds of formula (I), all six hydrogens, of R₄, which is dimethylamino, and all three hydrogens of each of R₅, R₇, and R₁₄, which are all a methoxy group, are enriched in deuterium. In a preferred embodiment, the compound of formula (I) is a compound of formula (II):

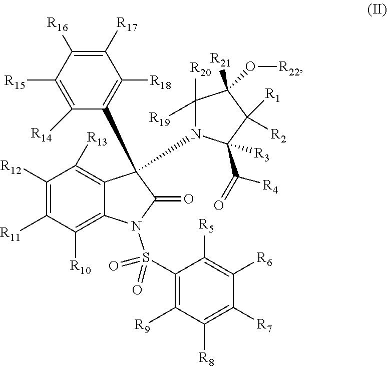

wherein R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, and R₂₂ are as defined above. The definitions and preferred embodiments of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, and R₂₂ of formula (II) are as given above for the compounds of formula (I).

In a more preferred embodiment, the compound of formula (I) is a compound of formula (III) or (IV):

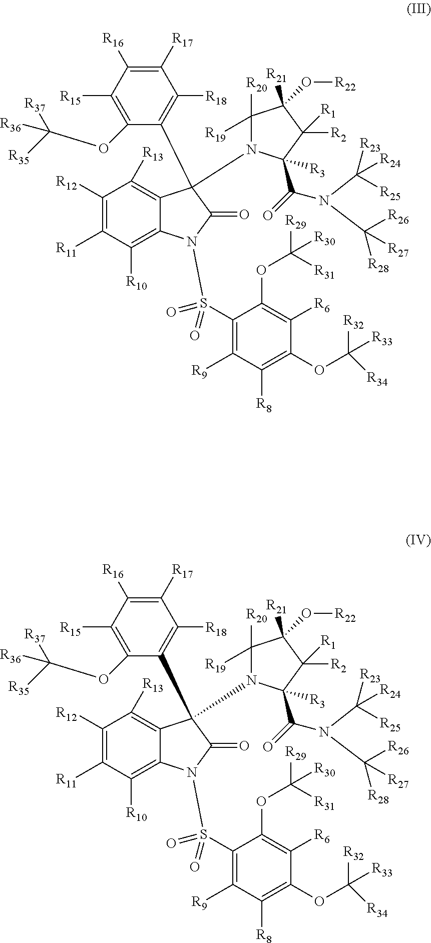

wherein R₁, R₂, R₃, R₆, R₈, R₉, R₁₀, R₁₁, R₁₂, R₁₃, R₁₅, R₁₆, R₁₇, R₁₈, R₁₉, R₂₀, R₂₁ and R₂₂ are as defined above, and R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ are hydrogen, provided that at least one hydrogen of R₁, R₂, R₃, R₆, R₈, R₉, R₁₀, R₁₁, R₁₂, R₁₃, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ is enriched in deuterium. The definitions and preferred embodiments of R₁, R₂, R₃, R₆, R₈, R₉, R₁₀, R₁₁, R₁₂, R₁₃, R₁₅, R₁₆, R₁₇, R₁₈, R₁₉, R₂₀, R₂₁ and R₂₂ of the compounds of formula (III) or (IV) are as given above for the compounds of formula (I) and (II), respectively.

In an even more preferred embodiment, the compound of formula (I) is a compound of formula (V):

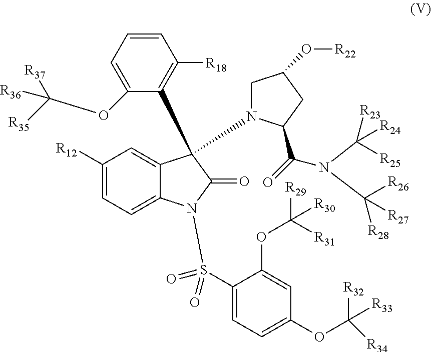

wherein R₂₂ is as defined above, preferably hydrogen, R₁₂ is as defined above, preferably chlorine, and at least one, preferably at least two or at least three, of R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ is enriched in deuterium.

Preferably, in the compound of formula (V)
a) R₂₃, R₂₄, R₂₅, R₂₆, R₂₇, and R₂₈ are enriched in deuterium,
b) R₂₉, R₃₀, R₃₁, R₃₂, R₃₃, and R₃₄ are enriched in deuterium, and/or
c) R₃₅, R₃₆ and R₃₇ are enriched in deuterium.

Particularly preferred embodiments of the compound of formula (I) according to the invention are the following compounds a) to g):
a) (2S,4R)1-[(3R)-5-chloro-1-[(2,4-dimethoxyphenyl) sulfonyl]-2,3-dihydro-3-(2-methoxyphenyl)-2-oxo-1H-indol-3-yl]-4-hydroxy-N,N-dimethyl-d6-2-pyrrolidinecarboxamide,
b) (2S,4R)1-[(3R)-5-chloro-1-[(2,4-dimethoxyphenyl) sulfonyl]-2,3-dihydro-3-(2-methoxy-d3-phenyl)-2-oxo-1H-indol-3-yl]-4-hydroxy-N,N-dimethyl-2-pyrrolidinecarboxamide,
c) (2S,4R)1-[(3R)-5-chloro-1-[(2,4-dimeth-d6-oxyphenyl) sulfonyl]-2,3-dihydro-3-(2-methoxyphenyl)-2-oxo-1H-indol-3-yl]-4-hydroxy-N,N-dimethyl-2-pyrrolidinecarboxamide,
d) (2S,4R)1-[(3R)-5-chloro-1-[(2,4-dimeth-d6-oxyphenyl) sulfonyl]-2,3-dihydro-3-(2-methoxy-d3-phenyl)-2-oxo-1H-indol-3-yl]-4-hydroxy-N,N-dimethyl-2-pyrrolidinecarboxamide,
e) (2S,4R)1-[(3R)-5-chloro-1-[(2,4-dimeth-d6-oxyphenyl) sulfonyl]-2,3-dihydro-3-(2-methoxy-phenyl)-2-oxo-1H-indol-3-yl]-4-hydroxy-N,N-dimethyl-d6-2-pyrrolidinecarboxamide,
f) (2S,4R)1-[(3R)-5-chloro-1-[(2,4-dimeth-oxyphenyl) sulfonyl]-2,3-dihydro-3-(2-methoxy-d3-phenyl)-2-oxo-1H-indol-3-yl]-4-hydroxy-N,N-dimethyl-d6-2-pyrrolidinecarboxamide, and
g) (2S,4R)1-[(3R)-5-chloro-1-[(2,4-dimeth-d6-oxyphenyl) sulfonyl]-2,3-dihydro-3-(2-methoxy-d3-phenyl)-2-oxo-1H-indol-3-yl]-4-hydroxy-N,N-dimethyl-d6-2-pyrrolidinecarboxamide.

Particularly preferred compounds are compounds a), b), c) and g). In one embodiment, one or more of compounds a), b), c), d), e), f) and/or g) are not encompassed.

The second aspect of the invention relates to a process for the preparation of a compound of formula (I):

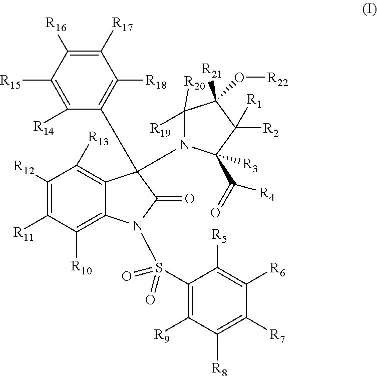

or a pharmaceutically acceptable salt or prodrug thereof, wherein the residues of the compound according to formula (I) are as defined above, in particular R₁, R₂, R₃, R₁₉, R₂₀, and R₂₁ are individually selected from the group consisting of hydrogen, C₁₋₄ alkyl, and C₁₋₄ alkoxy,
R₅, R₆, R₇, R₈, R₉, R₁₀, R₁₁, R₁₂, R₁₃, R₁₄, R₁₅, R₁₆, R₁₇, and R₁₈ are individually selected from the group consisting of hydrogen, halogen, C₁₋₄ alkyl, C₁₋₄ haloalkyl, C₁₋₄ alkoxy, and C₁₋₄ haloalkoxy,
R₄ is selected from the group consisting of C₁₋₄ alkylamino, C₁₋₄ haloalkylamino, di-C₁₋₄ alkylamino, di-C₁₋₄ haloalkylamino, and azetidin-1-yl,
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀, R₃₈ is selected from the group consisting of hydrogen, hydroxy and C₁₋₄ alkyl, R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and
integer n is 1 or 2, provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ is enriched in deuterium, wherein preferably the compound comprises at least an amount of deuterium as follows:
a) at least one hydrogen of R₄, which is selected from C₁₋₄ alkylamino or di-C₁₋₄ alkylamino, is enriched in deuterium, and/or
b) at least one hydrogen of R₅, R₇, or R₁₄, which are individually selected from C₁₋₄ alkyl or C₁₋₄ alkoxy, is enriched in deuterium, and
c) optionally at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium,
the process comprising a step of reacting at least one of the following compounds of formulae (VI), (VII), (VIII), or (IX)

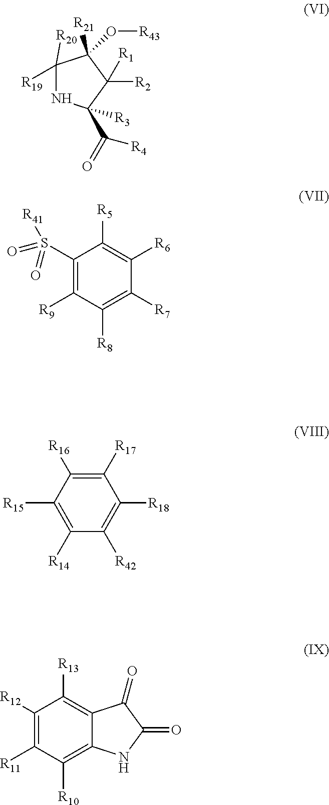

wherein R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁ are as defined above for formula (I), R₄₁ is a halogen or an activating ester residue, R₄₂ is a halogen, and R₄₃ is selected from the group consisting of hydrogen, C₁₋₄ alkyl, C₁₋₄ haloalkyl, —(CH₂)ₙ—CO—R₃₈, —CO—(CH₂)ₙ—NR₃₉R₄₀, (R₃₈, R₃₉ and R₄₀ are as defined above) and a hydroxyl protecting group, provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ of the compounds of formulae (VI), (VII), (VIII), or (IX) used is enriched in deuterium.

The preferred embodiments for the compounds according to formula (I) prepared by the process of the invention are those described above for the compound according to formula (I) according to the invention. In particular, the compounds according to formula (I) prepared by the process of the invention are preferably compounds according to formula (II), (III), (IV) or (V), respectively, including the respective definitions and preferred embodiments of the residues given above.

The preferred embodiments for the definitions of residues R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁ of the compounds of formulae (VI), (VII), (VIII), or (IX) are those as defined above for formula (I). Preferably
a) in the compound of formula (VI) at least one hydrogen, preferably all hydrogens, of R₄, which is selected from C₁₋₄ alkylamino or di-C₁₋₄ alkylamino, preferably dimethylamino, is enriched in deuterium, and preferably R₁, R₂, R₃, R₁₉, R₂₀ and R₂₁ are hydrogen, and R₄₃ is hydrogen or a hydroxyl protecting group,
b) in the compound of formula (VII) at least one hydrogen of R₅, or R₇, which are individually selected from C₁₋₄ alkyl or C₁₋₄ alkoxy, preferably methoxy, is enriched in deuterium, and preferably R₆, R₈, and Ry are hydrogen, and/or
c) in the compound of formula (VIII) at least one, preferably at least two or at least three, hydrogen of R₁₄, which is selected from C₁₋₄ alkyl or C₁₋₄ alkoxy, preferably methoxy, is enriched in deuterium, and preferably R₁₅, R₁₆, R₁₇, and R₁₈ are hydrogen.

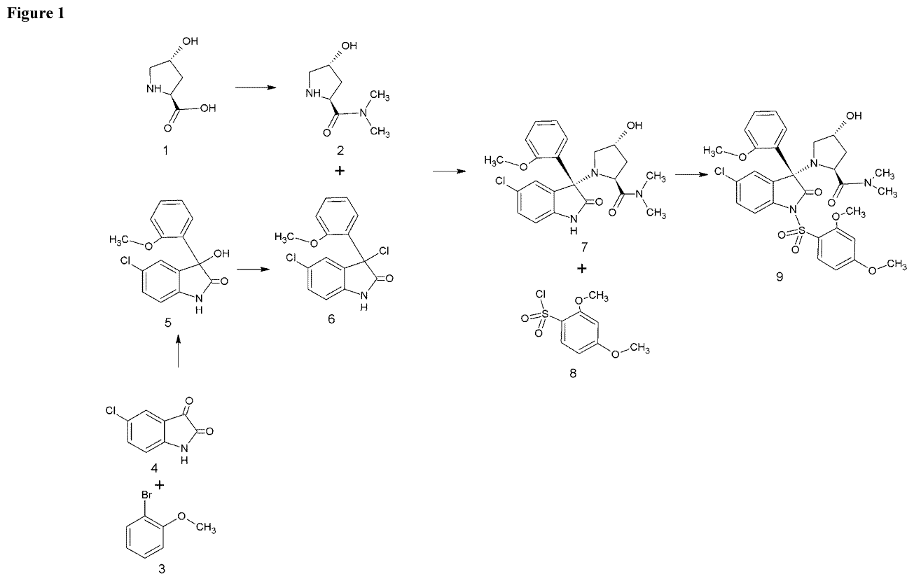

The compounds of formulae (VI), (VII), (VIII), or (IX) can be advantageously used in the preparation of a compound according to formula (I) of the present invention. The skilled person knows how to use the compounds of formulae (VI), (VII), (VIII), or (IX) as building blocks for preparing a compound according to formula (I). Suitable reaction pathways are exemplified in FIG. 1, the Examples as described herein, and are disclosed in U.S. Pat. No. 6,730,695B2. For Example, in accordance with FIG. 1, a compound according to formula (VIII) may be first reacted with a compound according to formula (IX). The product of the latter reaction may then be reacted, after optional conversion and/or activation, with a compound according to formula (VI), preferably followed by reaction with a compound of formula (VII). The latter reaction results in a compound according to formula (I).

The third aspect of the present invention relates to a pharmaceutical composition comprising a compound according to formula (I) as described above and a pharmaceutical acceptable carrier. The preferred embodiments of the compound according to formula (I) for the third aspect as those as described above for the first aspect. The pharmaceutical acceptable carrier is as known and established in the art, and as defined above. In the pharmaceutical compositions of the present invention for oral, which is preferred, sublingual, inhaled, subcutaneous, intramuscular, intravenous, transdermal, local or rectal administration, the active principles may be administered in unit administration forms, mixed with conventional pharmaceutical carriers, to animals and humans. The appropriate unit administration forms comprise oral forms such as tablets, gel capsules, powders, granules and oral solutions or suspensions, sublingual and buccal administration forms, aerosols, topical administration forms, implants, subcutaneous, intramuscular, intravenous, intranasal or intraocular administration forms and rectal administration forms.

In a fourth aspect the present invention relates to a compound according to formula (I) according to first aspect or a pharmaceutical composition according to the third aspect of the present invention for use as medicament.

In a fifth aspect, the present invention relates to a compound according to formula (I) according to first aspect or a pharmaceutical composition according to the third aspect of the present invention for the treatment or prophylaxis of disorders of the central nervous system, in particular of anxiety or depression. In other embodiments, the compounds can be used for the treatment or prevention of disorders of the cardiovascular system, for example hypertension; of the central nervous system, for example stress, anxiety, depression, obsessive compulsive disorder and panic attacks; of the renal system; of the gastric system as well as in the treatment of small cell lung cancers, of obesity; of type II diabetes, of insulin resistance, of hypertriglyceridemia; of atherosclerosis, of Cushing's Syndrome; of any pathology following stress and chronic stress states. Also encompassed by this embodiment is a method of treatment of these disorders by administering to a patient in need thereof an effective amount of a compound according to formula (I) as described above. In certain embodiments, the patient in need of said treatment has or shows symptoms of one of the above specified disorders, in particular disorders of the cardiovascular system, for example hypertension; of the central nervous system, for example stress, anxiety, depression, obsessive compulsive disorder and panic attacks; of the renal system; of the gastric system as well as in the treatment of small cell lung cancers, of obesity; of type II diabetes, of insulin resistance, of hypertriglyceridemia; of atherosclerosis, of Cushing's Syndrome; or of any pathology following stress and chronic stress states.

In certain embodiments, the use as a medicament according to a fourth aspect of the invention and the method of treatment according to the fifth aspect of the invention result in at least one effect selected from the group consisting of:

(1) decreased inter-individual variation in plasma levels of the compound or a metabolite thereof; (2) increased average plasma levels of the compound or decreased average plasma levels of at least one metabolite of the compound per dosage unit in a determined time frame; (3) decreased metabolism by at least one cytochrome P₄₅₀ in the subject; (4) decreased metabolism via at least one polymorphically-expressed cytochrome P₄₅₀ isoform in the subject; (5) at least one statistically-significantly improved outcome in the prophylaxis and/or treatment of the above mentioned disorders (6) an improved clinical effect during the treatment of the disorder; (7) prevention or delay of occurrence or recurrence of abnormal hepatic parameters; or (8) reduction or elimination of deleterious changes in any diagnostic hepatobiliary function endpoints, all of the above effects 1) to 8) as compared to the corresponding non-deuterated (non-isotopically enriched) compound.

In certain embodiments of the invention, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-deuterated compound.

Plasma levels of the compound as disclosed herein, or metabolites thereof, may be measured using the methods described by Li et al. Rapid Communications in Mass Spectrometry 2005, 19, 1943-1950; Jindal, et al., Journal of Chromatography, Biomedical Applications 1989, 493 (2), 392-7; Schwartz, et al., Biochemical Pharmacology 1966, 15 (5), 645-55; Mehvar, et al., Drug Metabolism and Disposition 1987, 15 (2), 250-5; Roberts et al., Journal of Chromatography, Biomedical Applications 1981, 226 (1), 175-82; and any references cited therein or any modifications made thereof.

Examples of diagnostic hepatobiliary function endpoints include, but are not limited to, alanine aminotransferase (“ALT”), and bilirubin.

Examples of cytochrome P450 isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51. In a preferred example, the P450 isoform is selected from the CYP3A enzymes, such as CYP3A4, CYP3A5, CYP3A5P1, CYP3A5P2, CYP3A7, for example CYP3A4 and CYP3A5, in particular CYP3A4.

The deuterated compounds of the invention show an increased degradation half-life compared to the corresponding non-deuterated compounds. E.g., the compounds of Examples 1, 2 and 3 show in increase in degradation half-life of +47%, +28% and +94%, respectively. Thus, the present invention preferably relates to derivatives of nelivaptan, in particular compounds according to formula (I) as described above, having an increased degradation half-life compared to the corresponding non-deuterated compounds, of at least 15%, more preferably at least 20%, e.g. at least 25%. The increase is considered as the percentage above 100%, relative to the degradation half-life of the corresponding non-deuterated compound, which is set to 100%. The degradation half-life is preferably determined by an in vitro human liver microsomal (HLM) stability assay, e.g. as described in Example 5.

A further aspect of the present invention relates to a compound according to formula (X):

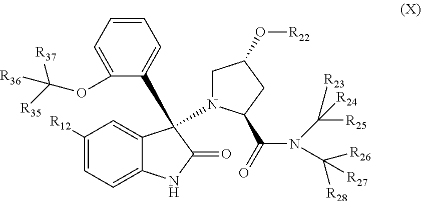

wherein R₁₂ is selected from the group consisting of hydrogen, halogen, C₁₋₄ alkyl, C₁₋₄ haloalkyl, C₁₋₄ alkoxy, and C₁₋₄ haloalkoxy preferably a methyl, trifluoromethyl or halogen, more preferably chlorine, R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀, R₃₈ is selected from the group consisting of hydrogen, hydroxy and C₁₋₄ alkyl, R₃₉ and Rao are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and integer n is 1 or 2, or R₂₂ is a hydroxyl protecting group, and R₂₃, R₂₄, R₂₅, R₂₆, R₂₇, R₂₈, R₃₅, R₃₆ and R₃₇ are hydrogen, provided that at least one, preferably at least two or at least three, of R₂₃, R₂₄, R₂₅, R₂₆, R₂₇, R₂₈, R₃₅, R₃₆ and R₃₇ is enriched in deuterium. Preferably, R₂₂ is hydrogen.

The definitions and further preferred embodiments of R₂₂, R₂₃, R₂₄, R₂₅, R₂₆, R₂₇, R₂₈, R₃₅, R₃₆, and R₃₇ of formula (X) are as given above for the compounds of formula (I) and formula (V). Preferably, in the compound of formula (X)
a) R₂₃, R₂₄, R₂₅, R₂₆, R₂₇, and R₂₈ are enriched in deuterium, and/or
b) R₃₅, R₃₆ and R₃₇ are enriched in deuterium.

The compounds of formula (X) are advantageous intermediates for the preparation of the compounds of formula (I) of the present invention, e.g. compound 7a, which can be used to obtain a compound 9a as shown in the Examples below.

### EXAMPLES

The following examples are for illustrative purposes only and do not limit the invention described above in any way.

The compounds as disclosed herein can be prepared by methods known to one of skill in the art and routine modifications thereof, and/or following procedures similar to those described in the Example section herein and routine modifications thereof, and/or procedures found in U.S. Pat. No. 6,730,695B2 and routine modifications thereof, which are hereby incorporated in their entirety, and references cited therein and routine modifications thereof.

Compounds as disclosed herein can in particular be prepared as shown in the general reaction scheme as shown in FIG. 1 and as described below, as well as routine modifications thereof.

### General Synthesis of the Compounds of the Invention

The compounds of the invention may be synthesized (by using deuterated starting materials) by a convergent synthesis as indicated in FIG. 1 which uses the following general building blocks:
Compound 1 (2S,4R)-4-hydroxy-N,N-dimethyl-2-pyrrolidinecarboxamide (trans-4-hydroxy-L-proline)
Compound 6 3-chloro-3-(2-methoxy-phenyl)-2-oxo-2,3-dihydro-1H-indole
Compound 8 2,4-dimethoxybenzenesulfonyl chloride.

The synthesis is following the general scheme as indicated in FIG. 1:

Compound 1 is converted into Compound 2, i.e. the dimethylamine derivative. The latter is reacted with Compound 6, which is available by reacting Compounds 3 and 4 to obtain Compound 5, which can be transferred to obtain Compound 6. The reaction of Compounds 2 and 6 results in Compound 7, which can be further reacted with Compound 8 to obtain the final product of Compound 9.

For the syntheses of the deuterated compounds of the invention, for the building blocks, e.g. Compounds 1, 6 and 8, deuterated starting materials can be used, which results in correspondingly deuterated end products. For Example, for Compound 2, deuterated dimethylamine (dimethylamine-d3) can be used, which is commercially available (e.g. as Dimethyl-d6-amin-hydrochlorid, CAS No. 53170-19-7). For Compound 3, i.e. o-bromoanisole, also a deuterated form thereof (o-bromoanisole-d3; CAS No. 100835-59-4) is commercially available. For Compound 8, 2,4-dimethoxybenzensulfonyl chloride or a deuterated form thereof, such as, 2,4-dimethoxy-d6-benzensulfonyl chloride can be used. Processes how to obtain deuterated building blocks for obtaining the compounds of the invention are known in the art (see e.g. Prakash et al., Chem. Soc. Rev., 2022, 51, 3123-3163).

The syntheses of the compounds of the invention has been exemplified herein below in context of the synthesis of (2S,4R)1-[(3R)-5-chloro-1-[(2,4-dimethoxyphenyl) sulfonyl]-2,3-dihydro-3-(2-methoxyphenyl)-2-oxo-1H-indol-3-yl]-4-hydroxy-N,N-dimethyl-d6-2-pyrrolidinecarboxamide.

### Example 1: Synthesis of (2S,4R)1-[(3R)-5-chloro-1-[(2,4-dimethoxyphenyl) sulfonyl]-2,3-dihydro-3-(2-methoxyphenyl)-2-oxo-1H-indol-3-yl]-4-hydroxy-N,N-dimethyl-d6-2-pyrrolidinecarboxamide

### Step 1: Preparation of Compound 2a (Deuterated Compound 2)

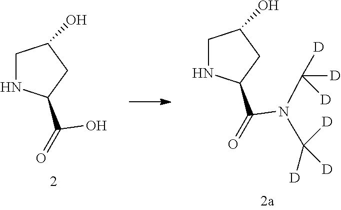 [SMILES: O=C(O)[C@@H]1C[C@@H](O)CN1 ; [2H]C([2H])([2H])N(C(=O)[C@@H]1C[C@@H](O)CN1)C([2H])([2H])[2H]]

40.0 g trans-4-Hydroxy-L-proline (2; 0.31 mol, 1.0 eq.) and 64.1 g NaHCO3 (0.76 mol, 2.5 eq.) were dissolved in 700 mL water and cooled to 0° C. 55.5 g (46.2 mL) Benzyl chloroformate (“Z—Cl”) (325 mmol, 1.05 eq.) in 200 mL toluene maintaining the temperature at 0° C. was added. The heterogenous reaction mixture was stirred overnight. The two layers were separated, and the aqueous layer was stripped of non-polar impurities by an extraction with diethyl ether (2×500 mL). The aqueous layer was then acidified to pH 4 with 10% aq. HCl and extracted with Chloroform/Isopropanol (7:3; 3×300 mL). After each extraction, the pH was again adjusted to pH 4. The combined organic layer was dried over MgSO₄, filtered, and concentrated under reduced pressure (viscous oil). The resulting Z-4-trans-L-hydroxyproline was dried under vacuum. Yield: 80.5 g. purity 97% (HPLC). Structure was confirmed by 1H-NMR in DMSO-d6.

68.0 g Z-4-trans-L-hydroxyproline (256 mmol, 1.0 eq.) was taken up in 1000 mL ethyl acetate and cooled down to 0° C. and 151 mL 1-propanephosphonic acid cyclic anhydride (50% in ethyl acetate; 256 mmol, 1.0 eq.) was added followed by 179 mL DIPEA (1.03 mol, 4.0 eq.) and the mixture was stirred for 5 min. Then, 20.2 g NMe₂-d6·HCl (0.9 eq.) was added batchwise and afterwards the reaction was stirred at rt (room temperature) overnight. The reaction mixture was evaporated, and the residue was redissolved in ethyl acetate (1 L) and washed with half-saturated Na₂CO₃-sol. (3×300 mL), dried over MgSO₄, filtered and the solvent removed in vacuum. The resulting Z-4-trans-L-hydroxyproline dimethyl-d6-amide was dried under vacuum. Yield: 34.0 g. purity ≥96% (HPLC). Structure confirmed by 1H-NMR in DMSO-d6.

31.0 g Z-4-trans-L-hydroxyproline dimethyl-d6-amide (104 mmol, 1.0 eq.) was taken up in 1 L of MeOH under N₂ and 3.10 g Pd/C was added. The atmosphere was exchanged by H₂ (3×) and stirred under H₂-balloon pressure. After 3 h the solution was filtered through a pad of celite and the celite bed was washed with MeOH. The solvent was removed in vacuum, the residue co evaporated with chloroform and dried under high vacuum. Result: 17.8 g Compound 2a; Purity ≥95%.

### Step 2: Preparation of Compound 6 Via Compound 5

### Step 2.1:

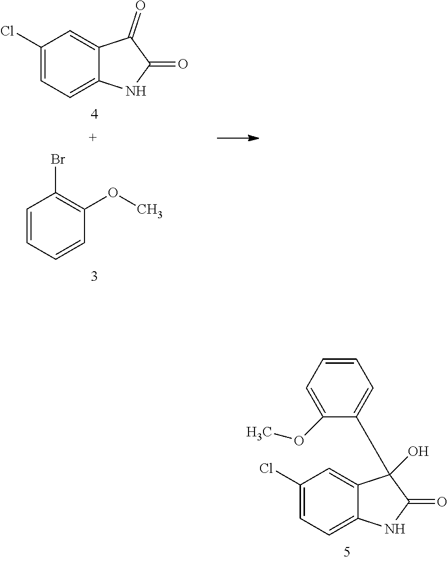

20.0 g 5-chloroisatin (4, 110.2 mmol, 1.0 eq.) was placed under N₂ to 300 mL of dry THF and cooled down to −78° C. In a 1000 mL 3-neck flask, 34.4 mL (51.6 g) 2-bromoanisole (3, 276 mmol, 2.5 eq.) was placed under N₂ in 200 mL dry THF and cooled down to −78° C. Under N₂, dropwise 110.2 mL n-BuLi (2.5 M; 276 mmol, 2.5 eq.) was added maintaining the temperature at ˜−78° C. After about 5 min, the solution was added to the 5-chloroisatin solution maintaining the temperature at −78° C. After 2 h, the reaction was quenched via addition of sat. NH₄Cl-sol. (400 mL) and extracted with ethyl acetate (4×300 mL). The combined organic layers were dried over MgSO₄, filtered and the solvent was removed in vacuo. The resulting material was treated with diethyl ether (200 mL) and the mixture was stirred for 5 min. The white solid was filtered off and dried under high vacuum.

Result: 30.2 g 3-hydroxy-3-(2-methoxy-phenyl)-2-oxo-2,3-dihydro-1H-indole 5, racemic, was obtained, purity >95% (HPLC)

### Step 2.2:

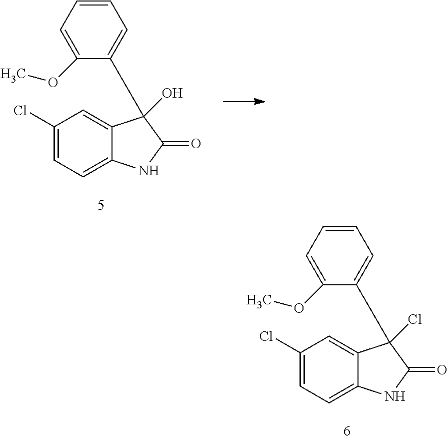 [SMILES: COc1ccccc1CC1(O)C(=O)Nc2ccc(Cl)cc21 ; COc1ccccc1CC1(Cl)C(=O)Nc2ccc(Cl)cc21]

A suspension of 22.0 g of the above 3-hydroxy-3-(2-methoxy-phenyl)-2-oxo-2,3-dihydro-1H indole, racemic, (5, 75.9 mmol, 1.0 eq) under N₂ in 300 mL dry dichloromethane was treated carefully with 6.18 mL pyridine (75.9 mmol, 1.0 eq.). The reaction mixture is cooled to 0° C. and 8.33 mL SOCl₂ (115 mmol, 1.5 eq.) was added dropwise via syringe. Then, the mixture was allowed to warm to rt over the course of 30 min. After 1 h, the reaction was quenched via addition of a small amount of crushed ice, extracted with dichloromethane (2×), dried over MgSO₄ and concentrated under vacuum. The crude residue was redissolved in dichloromethane (50 mL) and again concentrated under vacuum (repeated 3×). 21.5 g 3-chloro-3-(2-methoxy-phenyl)-2-oxo-2,3-dihydro-1H-indole, racemic compound 6 is isolated as a yellow solid and directly used in the subsequent step.

### Step 3: Coupling of Compound 2a to Compound 6 to Obtain Compound 7a (Deuterated Compound 7)

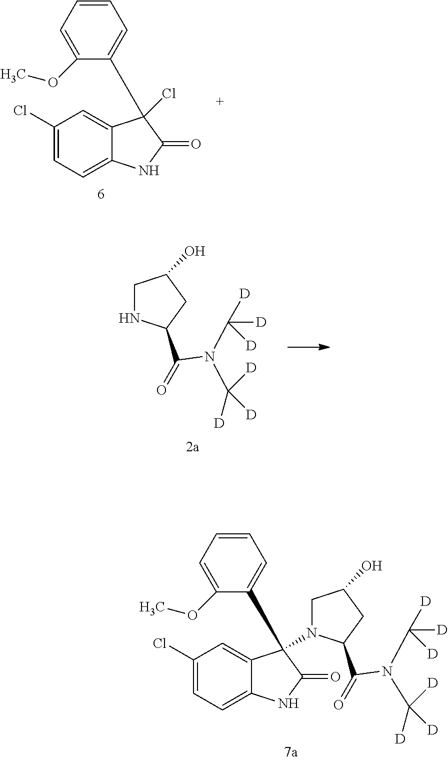 [SMILES: COc1ccccc1CC1(Cl)C(=O)Nc2ccc(Cl)cc21 ; [2H]C([2H])([2H])N(C(=O)[C@@H]1C[C@@H](O)CN1)C([2H])([2H])[2H] ; [2H]C([2H])([2H])N(C(=O)[C@@H]1C[C@@H](O)CN1[C@]1(c2ccccc2OC)C(=O)Nc2ccc(Cl)cc21)C([2H])([2H])[2H]]

Compound 6 (above) was suspended in 150 ml dry dichloromethane and cooled to 0° C. under N₂-atmosphere. Then, a mixture of 12.0 g Compound 2a (75.9 mmol, 1.0 eq.) and 10.7 mL NEt₃ (75.9 mmol, 1.0 eq.) in 150 mL dry dichloromethane was added slowly causing a moderate smoke formation. After 5 min, 21.3 mL NEt₃ (152 mmol, 2.0 eq.) was added and the reaction was warmed to room temperature overnight.

The reaction mixture was diluted with dichloromethane and washed with saturated NaHCO₃-solution (3×) and brine. The organic layers were concentrated under vacuum. Yield of racemate of Compound 7:31.5 g.

14 g of the above crude racemate of Compound 7a was dissolved in dichloromethane (10 mL) and purified by puriFlash5.020 using liquid injection. The column material used was 330 g irregular 50 μm SiO₂. This chromatographic step separated the diastereomers of compound 7a. Fractions were collected and analyzed by uHPLC.

Upon work-up 7.70 g of Compound 7a (2S,4R)1-[(3R)-5-chloro-2,3-dihydro-3-(2-methoxyphenyl)-2-oxo-1H-indol-3-yl]-4-hydroxy-N,N-dimethyl-d6-2-pyrrolidinecarboxamide is achieved. Purity was 99% and structure confirmed by 1H-NMR.

### Step 4: Synthesis of Compound 9a, (2S,4R)1-[(3R)-5-chloro-1-[(2,4-dimethoxyphenyl) sulfonyl]-2,3-dihydro-3-(2-methoxyphenyl)-2-oxo-1H-indol-3-yl]-4-hydroxy-N,N-dimethyl-d6-2-pyrrolidinecarboxamide

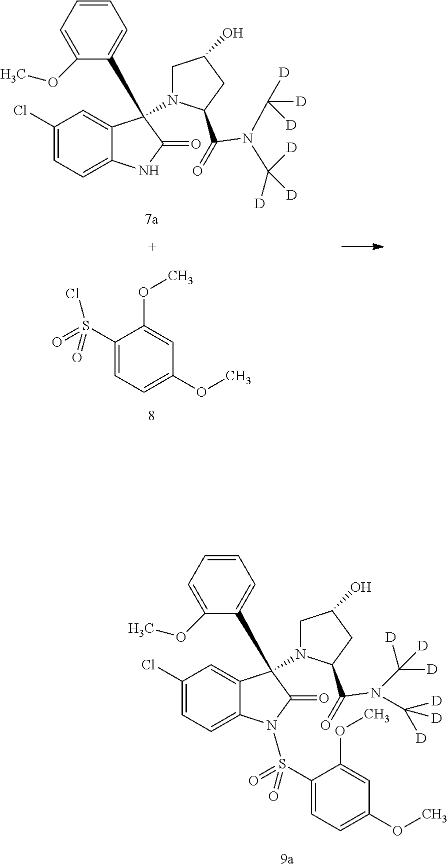 [SMILES: [2H]C([2H])([2H])N(C(=O)[C@@H]1C[C@@H](O)CN1[C@]1(c2ccccc2OC)C(=O)Nc2ccc(Cl)cc21)C([2H])([2H])[2H] ; COc1ccc(S(=O)(=O)Cl)c(OC)c1 ; [2H]C([2H])([2H])N(C(=O)[C@@H]1C[C@@H](O)CN1[C@]1(c2ccccc2OC)C(=O)N(S(=O)(=O)c2ccc(OC)cc2OC)c2ccc(Cl)cc21)C([2H])([2H])[2H]]

A suspension of 1.29 g NaH (32.2 mmol, 2.0 eq., 60% in mineral oil) in 60 mL dry dimethylformamide (DMF) under N₂ was cooled to 0° C. A solution of 7.00 g Compound 7a (above, 16.1 mmol, 1.0 eq.) in 60 mL dry DMF was added and the reaction was stirred for 5 min at 0° C. Then, a solution of 4.58 g Compound 8 (19.3 mmol, 1.2 eq.) in 60 mL dry DMF was added dropwise and the reaction was stirred at 0° C. until full conversion. After 1 h the reaction was quenched via addition of small amount of crushed ice (60 mL) followed by the addition of water (350 mL). The resulting mixture was extracted with ethyl acetate (3×300 mL). The combined organic layers were washed with water (3×) and brine, dried over MgSO₄ and concentrated under reduced pressure. The product was dissolved in dichloromethane and washed with water (2×) and brine (2×), dried over MgSO₄, filtered and the solvent was removed in vacuo. Crude product yield (Compound 9a): 9.0 g.

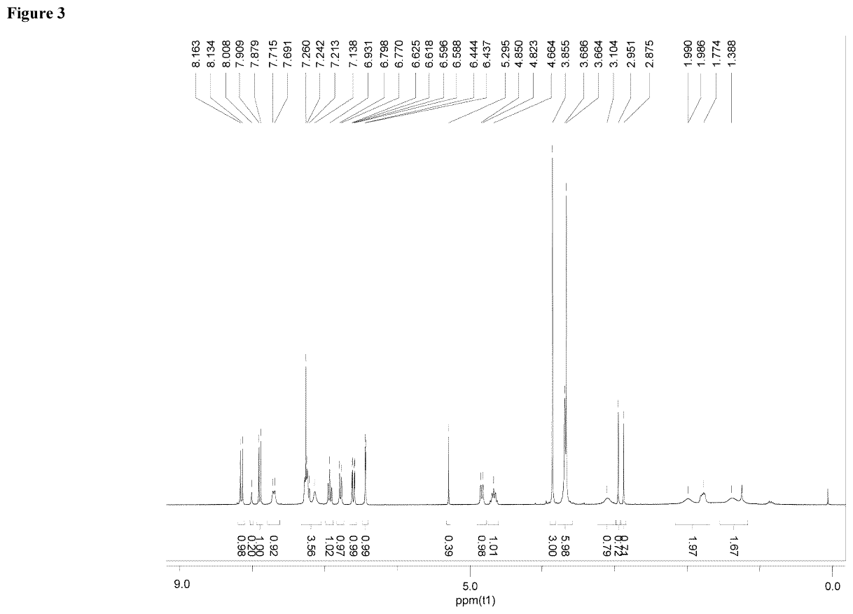

9.0 g of crude product was dissolved in dichloromethane (10 mL) and purified by puriFlash5.020 using liquid injection:
Column: FlashPure with 220 g Büchi irregular 50 μm SiO₂
Mobile phase: Ethyl acetate/MeOH (91:1)
Number of fractions: 2. Yield: 6.9 g
Analytical uHPLC: 3.01 min, M=636, >95%
1H-NMR as depicted in FIG. 3 shows the correct structure; purity >95%.

### Example 2: (2S,4R)1-[(3R)-5-chloro-1-[(2,4-dimethoxyphenyl) sulfonyl]-2,3-dihydro-3-(2-methoxy-d3-phenyl)-2-oxo-1H-indol-3-yl]-4-hydroxy-N,N-dimethyl-2-pyrrolidinecarboxamide

In analogy to the above-described synthesis of Example 1, (2S,4R)1-[(3R)-5-chloro-1-[(2,4-dimethoxyphenyl) sulfonyl]-2,3-dihydro-3-(2-methoxy-d3-phenyl)-2-oxo-1H-indol-3-yl]-4-hydroxy-N,N-dimethyl-2-pyrrolidinecarboxamide has been synthetized. Instead using a deuterated Compound 2 as in Example, compound 3a (a deuterated compound 3) as shown below was used, resulting in the title compound.

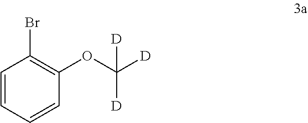 [SMILES: [2H]C([2H])([2H])Oc1ccccc1Br]

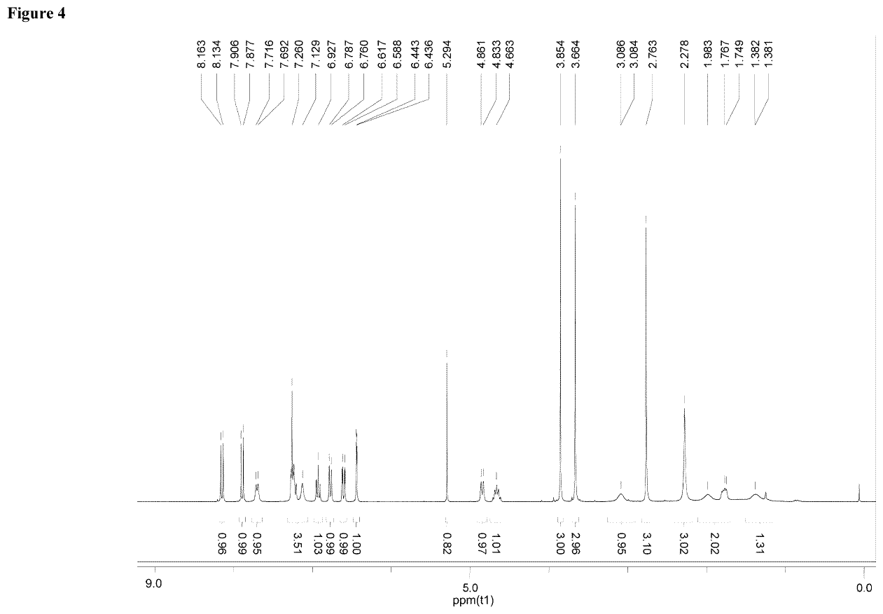

The 1H-NMR as depicted in FIG. 4 shows the correct structure of the title compound.

### Example 3: (2S,4R)1-[(3R)-5-chloro-1-[(2,4-dimeth-d6-oxyphenyl) sulfonyl]-2,3-dihydro-3-(2-methoxy-d3-phenyl)-2-oxo-1H-indol-3-yl]-4-hydroxy-N,N-dimethyl-d6-2-pyrrolidinecarboxamide

In analogy to the above-described synthesis of Examples 1 and 2, (2S,4R)1-[(3R)-5-chloro-1-[(2,4-dimeth-d6-oxyphenyl) sulfonyl]-2,3-dihydro-3-(2-methoxy-d3-phenyl)-2-oxo-1H-indol-3-yl]-4-hydroxy-N,N-dimethyl-d6-2-pyrrolidinecarboxamide has been synthetized using deuterated compounds 2, 3 and 8.

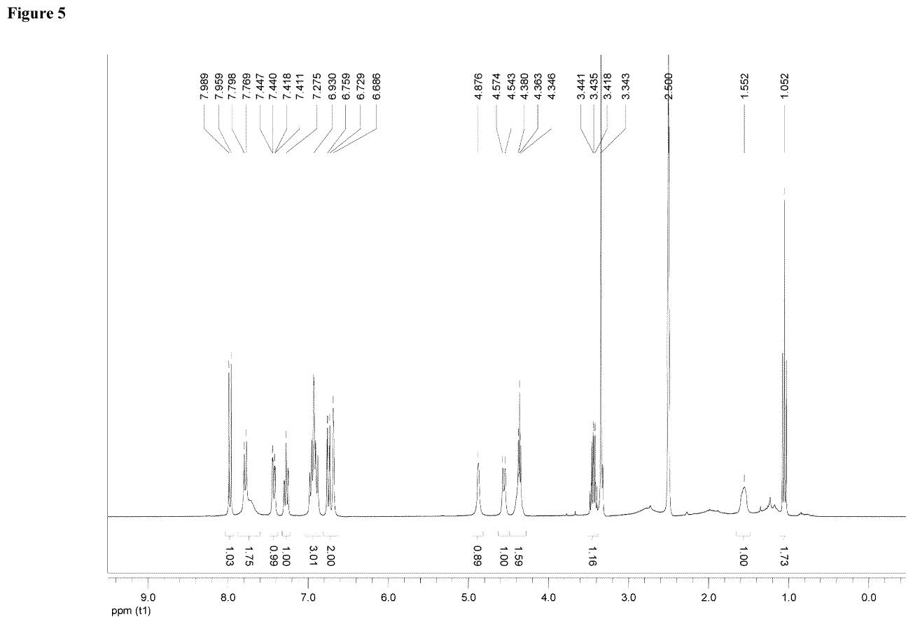

The 1H-NMR as depicted in FIG. 5 shows the correct structure of the title compound.

### Example 4

In analogy to the above-described synthesis, further deuterated compounds can be obtained using corresponding deuterated building blocks, such as
(2S,4R)1-[(3R)-5-chloro-1-[(2,4-dimethoxy-d6-phenyl) sulfonyl]-2,3-dihydro-3-(2-methoxy-phenyl)-2-oxo-1H-indol-3-yl]-4-hydroxy-N,N-dimethyl-2-pyrrolidinecarboxamide.

### Example 5

Changes in the metabolic properties of the compounds of the invention as compared to their non-isotopically enriched analogs can be shown using the following assays.

### Biological Activity Assay

### In Vitro Liver Microsomal Stability Assay

Liver microsomal stability assays are conducted with human liver microsomes at a final liver microsomal protein concentration of 0.1007 mg per ml. Test compounds are prepared as solutions in 0.01% DMSO, 0.25% acetonitril and 0.25% methanol. The test compound is added to the reaction mixture at a final concentration of 0.1 μM and pre-incubated with pooled liver microsomes in phosphate buffer (pH 7.4) for 10 min on plate shaker in a 37° C. incubator. The reaction is initiated by adding a NADPH-generating system and incubated for 0, 15, 30, 45, and 60 min. The reaction is stopped by transferring the incubation mixture to Stop Buffer solvent (acetonitrile/methanol). Samples are then mixed and centrifuged. The supernatant samples are loaded onto the LC/MS/MS for analysis. Samples are analyzed by LC/MS/MS using multiple reaction monitoring (MRM). The UPLC system consists of a binary LC pump with autosampler, a C-18 column, and a gradient. Conditions may be adjusted as necessary.

Four reference compounds are tested in each assay (Propranolol, imipramine, verapamil and terfenadine).

For data analysis, peak areas corresponding to the test compound are recorded. The compound remaining is calculated by comparing the peak area at each time point to time zero. The half-life is calculated from the slope of the initial linear range of the logarithmic curve of compound remaining (%) vs. time, assuming first order kinetics. In addition, the intrinsic clearance (Clint) is calculated from the half-life using the following equation:

$$CLint ( μ L / min / mg protein ) = 0.693 / ( T 1 / 2 \times Protein Conc . )$$

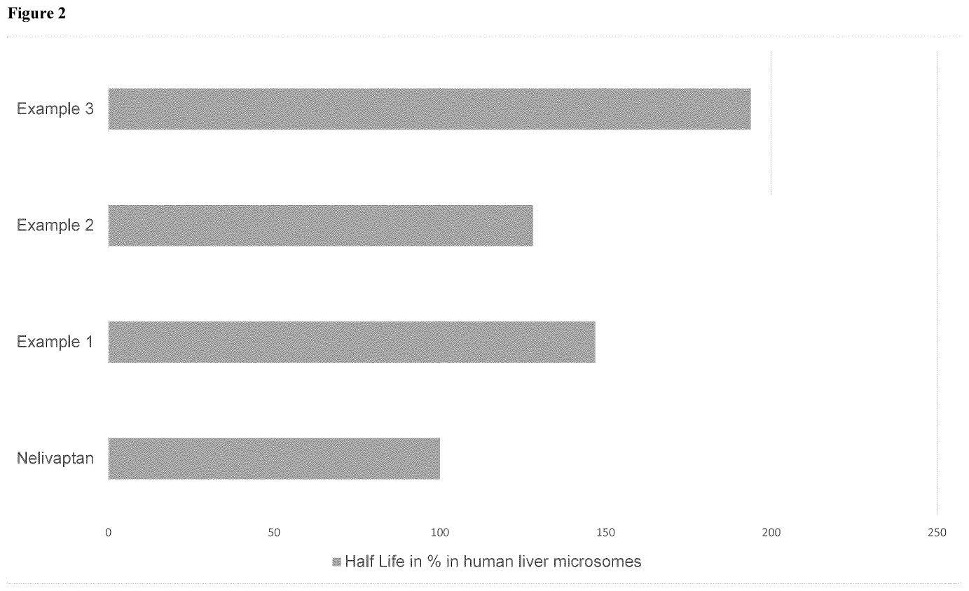

It has surprisingly been found that deuterium-enriched compounds of the invention as tested in this assay showed an increased degradation half-life as compared to the non-isotopically enriched drug nelivaptan. A comparison of the degradation half-lives of the compounds of Examples 1, 2 and 3 compared to the half live of the undeuterated nelivaptan is shown in Table 1 and FIG. 2.

TABLE 1
Results of in vitro human liver microsomal (HLM) stability assay

| | | | | % Increase of |
|---|---|---|---|---|
| | | | | human liver |
| | | % Compound | Half-life | microsomal |
| | | Remaining | (min) | degradation |
| Compound | Incubation | (Mean) | (Min.) | half-life |
| Nelivaptan | 0 | 100.0 | 3.2 | 0% |
| | 15 | 3.8 | | |
| | 30 | 0.4 | | |
| | 45 | 0.1 | | |
| | 60 | 0.03 | | |
| Example 1 | 0 | 100.0 | 4.7 | +46.88% |
| | 15 | 14.3 | | |
| | 30 | 1.2 | | |
| | 45 | 0.1 | | |
| | 60 | 0.03 | | |
| Example 2 | 0 | 100.0 | 4.1 | +28.125% |
| | 15 | 7.7 | | |
| | 30 | 0.4 | | |
| | 45 | 0.1 | | |
| | 60 | 0.02 | | |
| Example 3 | 0 | 100 | 6.2 | +93.75% |
| | 15 | 5.40 | | |
| | 30 | 0.39 | | |
| | 45 | 0.15 | | |
| | 60 | 0.13 | | |

### V1b Competitive Radioligand Binding Assay

Test compounds were tested for binding to the V1b human vasopressin receptor using a human V1b (agonist radioligand) Receptor Binding Assay, with the results summarized in Table 2. Test compounds were tested at several concentrations for IC50 or EC50 determination.

The detection method used was Scintillation counting. Ligand [3H]AVP was used with a Kd of 0.2 nM at concentrations of 0.2 nM. Incubation was for 120 min at RT. Control inhibitor used was Argipressin acetate and reference compound was AVP.

Compound binding was calculated as a % inhibition of the binding of a ligand specific for each target. The results are expressed as a percent of control specific binding

$$\frac{measured specific binding}{control specific binding} * 100$$

and as a percent inhibition of control specific binding

$$100 - ( \frac{measured specific binding}{control specific binding} * 100 )$$

obtained in the presence of the test compounds.

The IC50 values (concentration causing a half-maximal inhibition of control specific binding) and Hill coefficients (nH) were determined by non-linear regression analysis of the competition curves generated with mean replicate values using Hill equation curve fitting

$$Y = D + [ \frac{A - D}{1 + {( C / {C}_{50} )}^{n + 1}} ]$$

where Y=specific binding, A=left asymptote of the curve, D=right asymptote of the curve, C=compound concentration, C50=IC50, and nH=slope factor. This analysis was performed using software developed at Cerep (Hill software) and validated by comparison with data generated by the commercial software SigmaPlot® 4.0 for Windows® (© 1997 by SPSS Inc.). The inhibition constants (Ki) were calculated using the Cheng Prusoff equation

$${K}_{i} = \frac{{IC}_{50}}{( 1 + L / {K}_{D} )}$$

where L=concentration of ligand in the assay, and KD=affinity of the ligand for the receptor.

TABLE 2
Results of V1b competitive binding

| Compound | IC50 | Ki | nH |
|---|---|---|---|
| Nelivaptan | 1.5E−09M | 7.4E−10M | 0.9 |
| Example 1 | 2.3E−09M | 1.2E−09M | 1.3 |
| Example 2 | 1.9E−09M | 9.6E−10M | 1.1 |
| Example 3 | 2.0E−09M | 9.8E−10M | 1.1 |

All compounds tested demonstrate substantially the same inhibition efficiency.

Furthermore, an activity assay performed for the examples in context of the receptors V1a, OT receptors, and V2 in the agonistic mode showed for the compounds of Examples 1, 2 and 3 no significant difference in comparison to undeuterated nelivaptan.

### CONCLUSION

It has surprisingly been found that deuterium-enriched compounds of the invention as tested in these assays showed an increased degradation half-life as compared to the non-isotopically enriched drug nelivaptan, wherein their binding efficiency to the main receptor V1b does not seem to be impacted by said deuteration.

The following items are preferred embodiments of the present invention:

Item 1. A compound of formula (I):

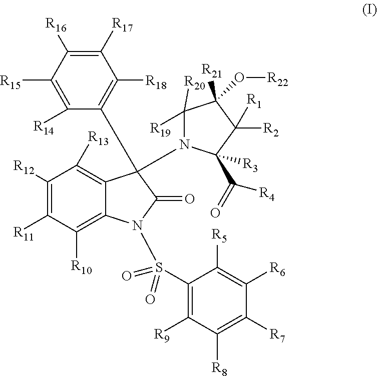

or a pharmaceutically acceptable salt or prodrug thereof, wherein:
R₁, R₂, R₃, R₁₉, R₂₀, and R₂₁ are individually selected from the group consisting of hydrogen, C₁₋₄ alkyl, and C₁₋₄ alkoxy,
R₅, R₆, R₇, R₈, R₉, R₁₀, R₁₁, R₁₂, R₁₃, R₁₄, R₁₅, R₁₆, R₁₇, and R₁₈ are individually selected from the group consisting of hydrogen, halogen, C₁₋₄ alkyl, C₁₋₄ haloalkyl, C₁₋₄ alkoxy, and C14 haloalkoxy,
R₄ is selected from the group consisting of C₁₋₄ alkylamino, C₁₋₄ haloalkylamino, di-C₁₋₄ alkylamino, di-C₁₋₄ haloalkylamino, and azetidin-1-yl,
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀,
R₃₈ is selected from the group consisting of hydrogen, hydroxy and C₁₋₄ alkyl,
R₃₉ and Rao are individually selected from the group consisting of hydrogen, C₁₋₄ alkyl, and C14 haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and integer n is 1 or 2,
provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ is enriched in deuterium.

Item 2. The compound according to item 1, wherein
a) at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ is enriched in deuterium,
b) at least one hydrogen of R₄, which is selected from C₁₋₄ alkylamino or di-C₁₋₄ alkylamino, preferably dimethylamino, is enriched in deuterium, and/or
c) at least one hydrogen of R₅, R₇, or R₁₄, which are individually selected from C₁₋₄ alkyl or C₁₋₄ alkoxy, preferably methoxy, is enriched in deuterium.

Item 3. The compound according to item 1 or 2, wherein
d) R₅, R₇, and R₁₄ are a methoxy group,
e) R₁₂ is a methyl, trifluoromethyl or halogen atom, preferably a chlorine atom,
f) R₂₂ is hydrogen, and/or
g) R₁, R₂, R₃, R₆, R₈, R₉, R₁₀, R₁₁, R₁₃, R₁₅, R₁₆, R₁₇, R₁₈, R₁₉, R₂₀, and R₂₁ are hydrogen.

Item 4. The compound of formula (I) according to any one of the preceding items, wherein at least one hydrogen, preferably all six hydrogens, of R₄, which is dimethylamino, and/or at least one hydrogen, preferably all three hydrogens, of R₅, R₇, or R₁₄, which are a methoxy group, is enriched in deuterium.

Item 5. The compound according to any one of the preceding items, which is a compound of formula (II):

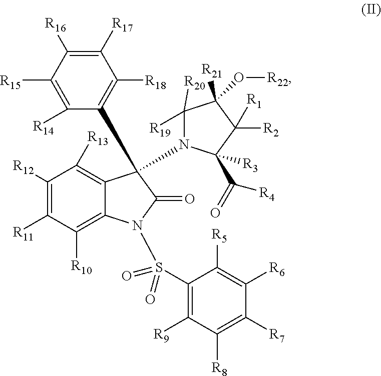

wherein R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, and R₂₂ are as defined in item 1.

Item 6. The compound according to any one of the preceding items, which is a compound of formula (III) or (IV):

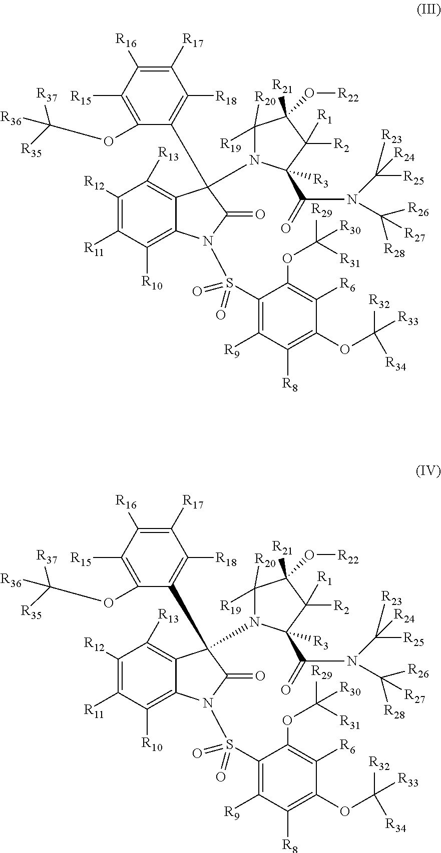

wherein R₁, R₂, R₃, R₆, R₈, R₉, R₁₀, R₁₁, R₁₂, R₁₃, R₁₅, R₁₆, R₁₇, R₁₈, R₁₉, R₂₀, R₂₁ and R₂₂ are as defined in item 1, and

R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ are hydrogen, provided that at least one hydrogen of R₁, R₂, R₃, R₆, R₈, R₉, R₁₀, R₁₁, R₁₂, R₁₃, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ is enriched in deuterium.

Item 7. The compound according to item 6, which is a compound of formula (V):

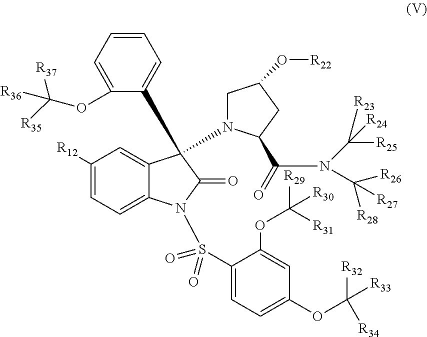

wherein R₂₂ is as defined in item 6, preferably hydrogen, R₁₂ is as defined in item 6, preferably chlorine, and at least one, preferably at least two or at least three, of R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ is enriched in deuterium.

Item 8. The compound according to item 7, wherein
a) R₂₃, R₂₄, R₂₅, R₂₆, R₂₇, and R₂₈ are enriched in deuterium,
b) R₂₉, R₃₀, R₃₁, R₃₂, R₃₃, and R₃₄ are enriched in deuterium, and/or
c) R₃₅, R₃₆ and R₃₇ are enriched in deuterium.

Item 9. The compound according to any one of the preceding items, wherein the enrichment in deuterium is at least 10%, at least 30%, at least 50%, at least 70%, at least 97%, at least 98%, or at least 99%.

Item 10. A process for the preparation of a compound of formula (I):

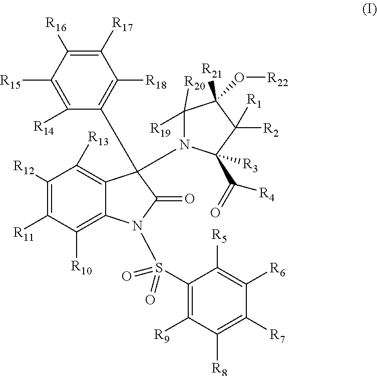

or a pharmaceutically acceptable salt or prodrug thereof, wherein:
R₁, R₂, R₃, R₁₉, R₂₀, and R₂₁ are individually selected from the group consisting of hydrogen, C₁₋₄ alkyl, and C₁₋₄ alkoxy,
R₅, R₆, R₇, R₈, R₉, R₁₀, R₁₁, R₁₂, R₁₃, R₁₄, R₁₅, R₁₆, R₁₇, and R₁₈ are individually selected from the group consisting of hydrogen, halogen, C₁₋₄ alkyl, C₁₋₄ haloalkyl, C₁₋₄ alkoxy, and C₁₋₄ haloalkoxy,
R₄ is selected from the group consisting of C₁₋₄ alkylamino, C₁₋₄ haloalkylamino, di-C₁₋₄ alkylamino, di-C₁₋₄ haloalkylamino, and azetidin-1-yl,
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀,
R₃₈ is selected from the group consisting of hydrogen, hydroxy and C₁₋₄ alkyl,
R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and
integer n is 1 or 2,
provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ is enriched in deuterium,
the process comprising a step of reacting at least one of the following compounds of formulae (VI), (VII), (VIII), or (IX)

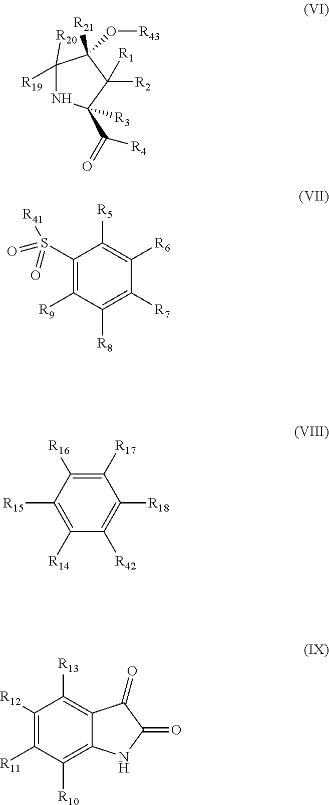

wherein R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉,
R₂₀, and R₂₁ are as defined for formula (I),
R₄₁ is a halogen or an activating ester residue,
R₄₂ is a halogen, and
R₄₃ is R₂₂ or a hydroxyl protecting group,
provided that at least one hydrogen of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, or R₂₁ of the compound of formulae (VI), (VII), (VIII), or (IX) used is enriched in deuterium.

Item 11. The process according to item 10, wherein
a) in the compound of formula (VI) at least one hydrogen of R₄, which is selected from C14 alkylamino or di-C₁₋₄ alkylamino, preferably dimethylamino, is enriched in deuterium, and preferably R₁, R₂, R₃, R₁₉, R₂₀ and R₂₁ are hydrogen and R₄₃ is hydrogen or a hydroxyl protecting group,
b) in the compound of formula (VII) at least one hydrogen of R₅, or R₇, which are individually selected from C₁₋₄ alkyl or C₁₋₄ alkoxy, preferably methoxy, is enriched in deuterium, and preferably R₆, R₈, and R₉ are hydrogen, and/or
c) in the compound of formula (VIII) at least one, preferably at least two or at least three, hydrogen of R₁₄, which is selected from C₁₋₄ alkyl or C₁₋₄ alkoxy, preferably methoxy, is enriched in deuterium, and preferably R₁₅, R₁₆, R₁₇, and R₁₈ are hydrogen.

Item 12. A pharmaceutical composition comprising a compound according to formula (I) according to any one of items 1 to 9 and a pharmaceutical acceptable carrier.

Item 13. A compound according to any of items 1 to 9 or the pharmaceutical composition according to item 12 for use as medicament.

Item 14. A compound according to any of items 1 to 9 or the pharmaceutical composition according to item 12 for use in the treatment or prophylaxis of disorders of the central nervous system, in particular of anxiety or depression.

Item 15. A compound according to formula (X):

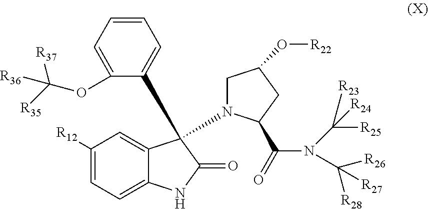

wherein
R₁₂ is selected from the group consisting of hydrogen, halogen, C₁₋₄ alkyl, C₁₋₄ haloalkyl, C₁₋₄ alkoxy, and C₁₋₄ haloalkoxy, preferably a methyl, trifluoromethyl or halogen, more preferably chlorine,
R₂₂ is selected from the group consisting of hydrogen, C₁₋₄ alkyl, C₁₋₄ haloalkyl, —(CH₂)ₙ—CO—R₃₈, and —CO—(CH₂)ₙ—NR₃₉R₄₀, R₃₈ is selected from the group consisting of hydrogen, hydroxy and C₁₋₄ alkyl, R₃₉ and R₄₀ are individually selected from the group consisting of hydrogen, C₁₋₄ alkyl, and C₁₋₄ haloalkyl, or R₃₉ and R₄₀, together with the nitrogen atom to which they are attached, constitute a heterocyclic group selected from the group consisting of: azetidin-1-yl, pyrrolidin-1-yl, piperid-1-yl, piperazin-1-yl, morpholin-4-yl and thiomorpholin-4-yl; and integer n is 1 or 2; or R₂₂ is a hydroxyl protecting group, and
R₂₃, R₂₄, R₂₅, R₂₆, R₂₇, R₂₈, R₃₅, R₃₆ and R₃₇ are hydrogen, provided that at least one, preferably at least two or at least three, of R₂₃, R₂₄, R₂₅, R₂₆, R₂₇, R₂₈, R₃₅, R₃₆ and R₃₇ is enriched in deuterium.

1. A compound of formula (IV): 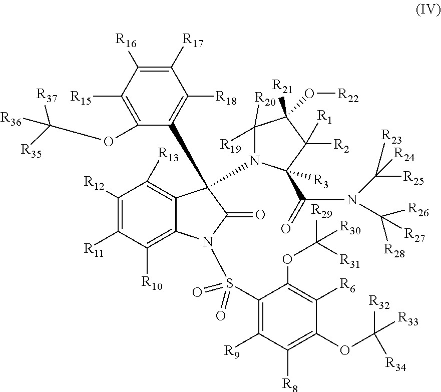
wherein R₁, R₂, R₃, R₆, R₈, R₉, R₁₀, R₁₁, R₁₃, R₁₅, R₁₆, R₁₇, R₁₈, R₁₉, R₂₀, R₂₁ and R₂₂ are hydrogen,
R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ are hydrogen,
R₁₂, is halogen, and
wherein the compound comprises at least an amount of deuterium as follows:
a) R₂₃, R₂₄, R₂₅, R₂₆, R₂₇, and R₂₈ are enriched in deuterium,
b) R₂₉, R₃₀, R₃₁, R₃₂, R₃₃, and R₃₄ are enriched in deuterium, and/or
c) R₃₅, R₃₆ and R₃₇ are enriched in deuterium.

2. The compound according to claim 1, wherein the compound comprises at least an amount of deuterium as follows:
a) R₂₃, R₂₄, R₂₅, R₂₆, R₂₇, and R₂₈ are enriched in deuterium,
b) R₂₉, R₃₀, R₃₁, R₃₂, R₃₃, and R₃₄ are enriched in deuterium, and
c) R₃₅, R₃₆ and R₃₇ are enriched in deuterium.

3. The compound according to claim 1, wherein the compound further comprises at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ enriched in deuterium.

4. The compound according to claim 1, which is a compound of formula (V): 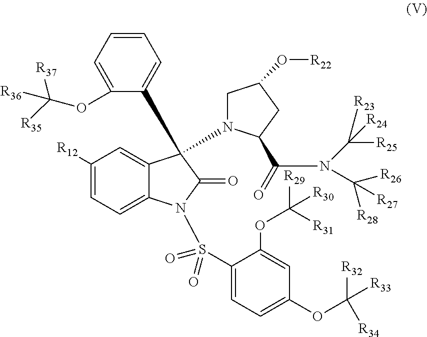
wherein
R₁₂ is chlorine.

5. The compound according to claim 1, wherein the enrichment in deuterium is at least 10%.

6. The compound according to claim 2, wherein the enrichment in deuterium is at least 10%.

7. The compound according to claim 3, wherein the enrichment in deuterium is at least 10%.

8. The compound according to claim 4, wherein the enrichment in deuterium is at least 10%.

9. The compound according to claim 1, wherein the enrichment in deuterium is selected from the group consisting of at least 30%, at least 50%, at least 70%, at least 97%, at least 98%, and at least 99%.

10. The compound according to claim 2, wherein the enrichment in deuterium is selected from the group consisting of at least 30%, at least 50%, at least 70%, at least 97%, at least 98%, and at least 99%.

11. The compound according to claim 3, wherein the enrichment in deuterium is selected from the group consisting of at least 30%, at least 50%, at least 70%, at least 97%, at least 98%, and at least 99%.

12. The compound according to claim 4, wherein the enrichment in deuterium is selected from the group consisting of at least 30%, at least 50%, at least 70%, at least 97%, at least 98%, and at least 99%.

13. A process for the preparation of a compound of formula (IV): 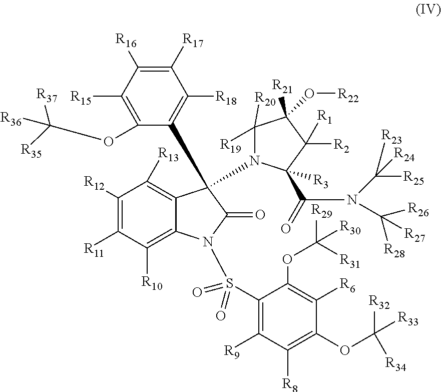
wherein R₁, R₂, R₃, R₆, R₈, R₉, R₁₀, R₁₁, R₁₃, R₁₅, R₁₆, R₁₇, R₁₈, R₁₉, R₂₀, R₂₁ and R₂₂ are hydrogen,
R₂₃, R₂₄, R₂₅, R₂₆, R₂₇, R₂₈, R₂₉, R₃₀, R₃₁, R₃₂, R₃₃, R₃₄, R₃₅, R₃₆ and R₃₇ are hydrogen, R₁₂, is halogen, and
wherein the compound comprises at least an amount of deuterium as follows:
a) R₂₃, R₂₄, R₂₅, R₂₆, R₂₇, and R₂₈ are enriched in deuterium,
b) R₂₉, R₃₀, R₃₁, R₃₂, R₃₃, and R₃₄ are enriched in deuterium, and/or
c) R₃₅, R₃₆ and R₃₇ are enriched in deuterium,
the process comprising a step of reacting at least one of the following compounds of formulae (VI), (VII), (VIII), or (IX) 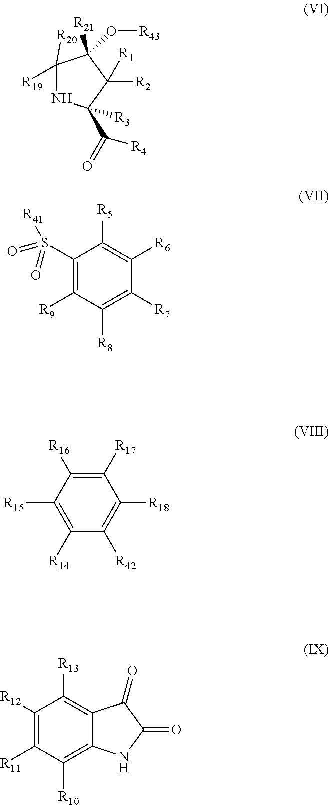
wherein R₁, R₂, R₃, R₆, R₈, R₉, R₁₀, R₁₁, R₁₂, R₁₃, R₁₅, R₁₆, R₁₇, R₁₈, R₁₉, R₂₀, and R₂₁ are as defined for formula (IV),
R₄ is dimethylamino,
R₅, R₇ and R₁₄ are methoxy,
R₄₁ is a halogen or an activating ester residue,
R₄₂ is a halogen, and
R₄₃ is hydrogen or a hydroxyl protecting group, wherein
a) in the compound of formula (VI) all hydrogens of R₄ are enriched in deuterium,
b) in the compound of formula (VII) all hydrogens of R₅ and/or R₇ are enriched in deuterium, and/or
c) in the compound of formula (VIII) three hydrogens of R₁₄, are enriched in deuterium.

14. The process according to claim 13, wherein the compound of formula (IV) further comprises at least one hydrogen of R₁, R₂, R₃, R₆, R₈, R₉, R₁₀, R₁₁, R₁₃, R₁₅, R₁₆, R₁₇, R₁₈, R₁₉, R₂₀, or R₂₁ enriched in deuterium.

15. A pharmaceutical composition comprising a compound according to claim 1 and a pharmaceutical acceptable carrier.

16. A composition comprising a compound according to claim 1 and a carrier or diluent.

17. A method for the treatment or prophylaxis of disorders of the central nervous system in a patient in need thereof comprising administering a compound according to claim 1 to the patient.

18. The method according to claim 17, wherein the disorder of the central nervous system comprises anxiety or depression.

19. A method for the treatment or prophylaxis of disorders of the central nervous system in a patient in need thereof comprising administering the pharmaceutical composition according to claim 15 to the patient.

20. The method according to claim 19, wherein the disorder of the central nervous system comprises anxiety or depression.
